(19) United States
(12) Patent Application Publication
(10) Pub. No.: US 2026/0354781 A1
(43) Pub. Date: Nov. 12, 2026

(54) SYSTEM AND METHOD FOR THREE-DIMENSIONAL ANATOMICAL RECONSTRUCTION FROM SEQUENTIAL FLUOROSCOPIC VIEWS

(71) Applicant: See All AI Inc., Nashua, NH (US)

(72) Inventors: Eugene Alma GREGERSON, North Salt Lake, UT (US); Eric David MAJOR, Purcellville, VA (US)

(73) Assignee: See All AI Inc., Nashua, NH (US)

(21) Appl. No.: 19/672,195

(22) Filed: May 8, 2026

Related U.S. Application Data
(60) Provisional application No. 63/803,342, filed on May 9, 2025.

(51) Int. Cl. A61B 6/00; A61B 6/03; A61B 6/58; G06T 7/00; G06T 7/246; G06T 15/08; G06V 10/25; G06V 10/80

(52) U.S. Cl. CPC A61B 6/487; A61B 6/035; A61B 6/485; A61B 6/547; A61B 6/585; G06T 7/0012; G06T 7/246; G06T 15/08; G06V 10/25; G06V 10/806; G06T 2207/10064; G06T 2207/10121; G06T 2207/30204

(57) ABSTRACT

A system reconstructs a three-dimensional volumetric image of an extended anatomical region exceeding the native field of view of a fluoroscopic detector. A fluoroscopic image acquisition system captures a sequence of N fluoroscopic frames, where N>4, during one or more continuous translational sweeps along the anatomical region, with successive frames having at least partial spatial overlap. A first fiducial marker is mounted to the imaging system and a second fiducial marker is affixable to the patient. A spatial tracking system generates six-degree-of-freedom positional data for at least one frame, and projection matrices for remaining frames are derived by direct tracking measurement, interpolation, image-based inter-frame registration, or combinations thereof. A processor produces one or more two-dimensional representations from the fluoroscopic frames, back-projects the representations into one or more three-dimensional voxel grids, and processes the back-projected representations using a trained three-dimensional deep learning network to generate a volumetric output automatically registered to the patient coordinate system without requiring a preoperative CT scan.

### CROSS REFERENCE TO RELATED APPLICATIONS

This application claims the benefit of priority under 35 U.S.C. § 119(e) to U.S. Provisional Application No. 63/803,342, entitled “System and Method for Three Dimensional Anatomical Reconstruction from Sequential Fluoroscopic Views,” filed May 9, 2025, the disclosure of which is incorporated herein by reference in its entirety.

### TECHNICAL FIELD

The disclosure relates to medical imaging, and more specifically, to the use of artificial intelligence in conjunction with fluoroscopic imaging to reconstruct CT-like three-dimensional (3D) volumes of human anatomy that span an extended anatomical region that exceeds the native field of view of conventional fluoroscopy detectors.

### BACKGROUND

Intraoperative imaging plays a fundamental role in modern surgical and interventional practice, providing clinicians with real-time visual guidance during procedures across a broad range of clinical specialties. Among the various intraoperative imaging modalities, fluoroscopy is the most widely used due to its relative affordability, portability, and availability across virtually all surgical environments. However, conventional fluoroscopy produces only two-dimensional projection images, and the spatial extent of any single image is inherently limited by the physical dimensions of the imaging detector. When the anatomical region of clinical interest exceeds the detector's field of view, the clinician must acquire multiple separate images at different positions, a process that introduces spatial discontinuities between images, increases cumulative radiation exposure to the patient and surgical staff, extends procedural time, and yields no three-dimensional volumetric information about the imaged anatomy.

Three-dimensional intraoperative imaging systems capable of producing volumetric data do exist; however, they generally entail significant practical barriers that limit their widespread adoption. Such systems tend to be substantially more expensive than standard fluoroscopic equipment, are unavailable in many surgical settings, and often deliver higher radiation doses to produce their volumetric reconstructions. Furthermore, the volumetric coverage of these systems is typically constrained to a limited spatial region that may be insufficient for anatomical structures extending over large portions of the body. In many conventional surgical navigation workflows, the generation of three-dimensional guidance data also depends upon imaging studies acquired prior to surgery and upon manual procedures to align the patient's anatomy with those preexisting datasets, adding further cost, complexity, and radiation burden. These barriers collectively restrict the availability of three-dimensional intraoperative imaging and navigation across the diverse range of clinical environments and procedures in which such capabilities would provide meaningful benefit.

Accordingly, a need exists for improved systems and methods capable of generating three-dimensional volumetric images of anatomical regions that exceed the field of view achievable in a single conventional image, using widely available and cost-effective imaging equipment, with clinically acceptable radiation exposure, and without dependence upon preoperative imaging studies or manual registration procedures.

### SUMMARY

In one aspect, the present disclosure provides a system for reconstructing a three-dimensional volumetric image of an extended anatomical region of a patient exceeding the native field of view of a fluoroscopic detector. The system comprises a fluoroscopic image acquisition system configured to capture a sequence of N fluoroscopic frames during one or more continuous translational sweeps along an extended anatomical region of interest, wherein N is greater than (>) 4, and wherein successive frames have at least partial spatial overlap. The system further comprises a first fiducial marker rigidly mounted to the fluoroscopic image acquisition system, a second fiducial marker affixable to the patient proximate the anatomical region of interest, and a spatial tracking system comprising at least one spatial sensing device and configured to track both the first and second fiducial markers during at least a portion of the sequence of fluoroscopic frames, thereby generating six-degree-of-freedom positional data for at least one frame in the sequence. The positional data is usable, in combination with intrinsic camera parameters of the fluoroscopic image acquisition system, to derive a projection matrix, wherein a projection matrix for each of the remaining frames is derived from the positional data by at least one of direct tracking measurement, interpolation between tracked frames, image-based inter-frame registration, or any combination thereof. The system further comprises a processor configured to execute a trained neural network model that produces, from the N fluoroscopic frames, one or more two-dimensional representations, each two-dimensional representation being selected from a raw fluoroscopic image, a composited panoramic image formed by spatially registering and blending a plurality of sequential frames, or learned feature maps extracted by a trained two-dimensional deep learning network operating on individual fluoroscopic frames or on a composited panoramic image. The trained neural network model back-projects the one or more two-dimensional representations into one or more three-dimensional voxel grids using projection matrices derived from the positional data, and produces a volumetric output by processing the back-projected representations through a trained three-dimensional deep learning network. The volumetric output spans a spatial extent, in at least one dimension, exceeding the field of view of any single fluoroscopic frame, and is automatically registered to a patient coordinate system via spatial tracking measurements of the first and second fiducial markers, the registration being independent of any preoperative volumetric imaging study of the patient and any manual intraoperative landmark-correspondence step.

In some embodiments, the two-dimensional representation comprises multi-resolution feature maps extracted by a deep learning encoder network producing features at a plurality of spatial resolution levels with respective downsampling factors.

In some embodiments, the deep learning encoder network is selected from the group comprising a ResNet-family convolutional neural network, a VGG-family network, an EfficientNet, a DenseNet, a MobileNet, a ConvNeXt, a ViT, a DeiT, a Swin Transformer, a PVT, a CoAtNet, a MaxViT, and a hybrid CNN-transformer architecture, producing feature maps at a plurality of resolution levels.

In some embodiments, the two-dimensional representation comprises the raw fluoroscopic image without learned two-dimensional encoding. The present disclosure therefore supports both learned and identity-based two-dimensional representations while preserving the same geometry-aware downstream reconstruction concept.

In some embodiments, the back-projection operates at multiple spatial scales, producing a plurality of three-dimensional cubes at progressively coarser resolutions, wherein different two-dimensional feature levels are selectively routed to different three-dimensional scales via a configurable feature-to-scale mapping.

In some embodiments, the three-dimensional deep learning network comprises a U-Net with both a three-dimensional encoder and a three-dimensional decoder, wherein the encoder processes multi-scale back-projected cubes from finest to coarsest resolution with skip connections, and the decoder upsamples with symmetric skip connections.

In some embodiments, the back-projection produces a single three-dimensional voxel grid at a single spatial scale, and the three-dimensional deep learning network comprises a monolithic three-dimensional encoder-decoder network selected from the group comprising a 3D convolutional neural network, a MONAI UNet, a Vision Transformer, a UNETR, a SwinUNETR, a SegResNet, and a hybrid CNN-transformer architecture, the monolithic network receiving the single back-projected voxel grid and performing both encoding and decoding to produce the volumetric output.

In some embodiments, the volumetric output simultaneously comprises a CT-like reconstruction volume, a multi-class tissue segmentation volume, a binary metal segmentation volume, and an anatomical part segmentation volume, each produced by a separate output head from a shared three-dimensional feature volume. Representative tissue classes include bone, soft tissue, air, disc, and spinal canal. Representative anatomical-part classes include vertebral body, canal, disc, posterior elements, processes, or other anatomical structures. The present disclosure therefore supports a shared latent volumetric representation from which multiple clinically useful volumetric outputs are generated.

In some embodiments, each fluoroscopic frame contains a radiopaque calibration device comprising a plurality of radiopaque markers at known three-dimensional coordinates, and per-frame intrinsic and extrinsic camera parameters are computed from detected marker features by establishing two-dimensional-to-three-dimensional point correspondences and solving the perspective-n-point problem.

In some embodiments, radiopaque calibration device artifacts are digitally removed from each frame prior to computation of the two-dimensional representation.

In some embodiments, the radiopaque calibration device comprises a radiopaque geometry selected from the group consisting of a wire-based device having crossover features or gap features, a spherical-marker device having centroid features, a coded-marker arrangement, a grid pattern, a plate pattern, and combinations thereof, the radiopaque geometry providing known three-dimensional feature locations visible in the fluoroscopic frame for per-frame inline calibration. The present disclosure therefore supports per-frame inline calibration without requiring a separate pre-imaging calibration procedure.

In some embodiments, the processor is further configured to, prior to back-projection, composite the sequential fluoroscopic frames from each sweep orientation into a panoramic image by spatially registering each frame using its per-frame projection matrix and blending overlapping regions using spatially weighted averaging, and to back-project the panoramic image into a single extended three-dimensional voxel grid spanning the full swept anatomy without sub-volume tiling.

In some embodiments, the one or more continuous translational sweeps comprise at least one sweep along an axis that is not parallel to the patient's longitudinal axis, and the volumetric output spans a spatial extent along the sweep direction exceeding the field of view of any single fluoroscopic frame. Thus, the present disclosure is not limited to cranial-caudal sweeps along the spine, but also includes transverse, anterior-posterior, oblique, and multi-axis sweeps for wide anatomy such as the pelvis, head, bilateral shoulders, bilateral knees, bilateral feet, and other anatomy whose in-plane extent exceeds the detector field of view.

In some embodiments, the three-dimensional deep learning network comprises a generative model selected from the group consisting of a generative adversarial network, a diffusion model, a variational autoencoder, and a neural radiance field decoder. Such generative architectures enable the system to synthesize high-fidelity volumetric detail from limited angular diversity inherent in translational sweep geometry. The technical effect of employing a generative reconstruction model includes improved volumetric image quality in regions where translational projections alone provide limited tomographic sampling, thereby supporting extended-field-of-view reconstruction without requiring orbital cone-beam acquisition.

In some embodiments, the spatial tracking system generates six-degree-of-freedom positional data for every frame in the sequence, each frame having a temporally synchronized pose measurement. Per-frame tracking provides high geometric accuracy for the projection matrices used in back-projection, and the technical effect includes reduced registration uncertainty and improved volumetric fidelity in the extended-field reconstruction.

In an aspect, the present disclosure provides a system for generating a three-dimensional volumetric image of an extended anatomical region exceeding the native field of view of a fluoroscopic detector. The system comprises a fluoroscopic image acquisition system configured to capture a sequence of N fluoroscopic frames during one or more continuous translational sweeps along an extended anatomical region of interest, wherein N is greater than (>) 4 and successive frames have at least partial spatial overlap. The system further comprises a processor configured to composite sequential fluoroscopic frames from at least one sweep orientation into a panoramic image by spatially registering and blending a plurality of sequential frames, back-project the panoramic image into a single extended three-dimensional voxel grid spanning the full swept anatomy, and produce a volumetric output by processing the single extended three-dimensional voxel grid through a trained three-dimensional deep learning network. The volumetric output spans a spatial extent exceeding the field of view of any single fluoroscopic frame.

In some embodiments, the panoramic image is processed by a two-dimensional encoder network to produce panoramic feature maps prior to back-projection, thereby enabling the encoder network to learn long-range anatomical relationships spanning the full extent of the swept anatomy.

In some embodiments, the spatial registration of sequential fluoroscopic frames for compositing the panoramic image is determined from image content by detecting and matching features in overlapping regions between adjacent frames and estimating inter-frame geometric transformations, rather than from per-frame spatial tracking measurements.

In some embodiments, a six-degree-of-freedom pose is captured by a spatial tracking system for only a single reference frame in the sweep sequence, and projection matrices for all remaining frames are derived by composing the image-derived inter-frame geometric transformations into a cumulative transform chain from the single reference pose. In this way, the present disclosure includes both tracker-based panoramic stitching and image-based panoramic stitching for long-field fluoroscopic reconstruction.

In some embodiments, each fluoroscopic frame is acquired as a digital image in a format selected from the group consisting of a DICOM image, a cine digital sequence, a raw detector output, and any other digital image format, the digital image preserving at least 10 bits of the bit depth of the fluoroscopic detector.

In an aspect, the present disclosure provides a method for generating a three-dimensional volumetric image of an extended anatomical region that exceeds the native field of view of a fluoroscopic detector. The method comprises acquiring a sequence of N fluoroscopic frames, wherein N is greater than 4, during one or more continuous translational sweeps along an extended anatomical region of interest, wherein successive frames have at least partial spatial overlap, the fluoroscopic frames acquired in any image format including native digital formats or digitized analog video. The method further comprises tracking, via a spatial tracking system comprising at least one spatial sensing device, a first fiducial marker mounted to the fluoroscopic image acquisition system and a second fiducial marker affixed to a patient, thereby generating six-degree-of-freedom positional data for at least one frame in the sequence, the positional data being usable, in combination with intrinsic camera parameters of the fluoroscopic image acquisition system, to derive a projection matrix for each of the N frames by at least one of direct tracking measurement, interpolation between tracked frames, image-based inter-frame registration, or any combination thereof. The method further comprises producing, from the N fluoroscopic frames, one or more two-dimensional representations, each two-dimensional representation being selected from a raw fluoroscopic image, a composited panoramic image formed by spatially registering and blending a plurality of sequential frames, or learned feature maps extracted by a trained two-dimensional deep learning network operating on individual fluoroscopic frames or on a composited panoramic image. The method further comprises back-projecting the one or more two-dimensional representations into one or more three-dimensional voxel grids using projection matrices derived from the positional data, producing a volumetric output by processing the back-projected representations through a trained three-dimensional deep learning network, the volumetric output spanning a spatial extent, in at least one dimension, exceeding the field of view of any single fluoroscopic frame, and automatically registering the volumetric output to a patient coordinate system via spatial tracking measurements of the first and second fiducial markers, the registration being independent of any preoperative volumetric imaging study of the patient and any manual intraoperative landmark-correspondence step.

In some embodiments of the method, the volumetric output is reconstructed as a plurality of tiled sub-volumes blended in overlap regions using Gaussian-weighted averaging.

In some embodiments, the method further comprises, prior to back-projection, compositing the sequential fluoroscopic frames from each sweep orientation into a panoramic image by spatially registering each frame and blending overlapping regions, and back-projecting the panoramic image into a single extended three-dimensional voxel grid spanning the full swept anatomy.

In some embodiments, the spatial registration of sequential frames for compositing the panoramic image is determined from image content by detecting and matching features in overlapping regions between adjacent frames rather than from per-frame spatial tracking measurements, and a six-degree-of-freedom pose is captured for only a single reference frame in the sweep sequence.

In some embodiments, each fluoroscopic frame contains a radiopaque calibration device comprising a plurality of radiopaque markers at known three-dimensional coordinates, and the method further comprises computing per-frame intrinsic and extrinsic camera parameters from detected marker features by establishing two-dimensional-to-three-dimensional point correspondences and solving the perspective-n-point problem.

In some embodiments, radiopaque calibration device artifacts are digitally removed from each frame prior to producing the two-dimensional representations.

In some embodiments, the method further comprises training the trained three-dimensional deep learning network on pairs of CT volumes and simulated digitally reconstructed radiographs generated via polychromatic X-ray simulation with clinically matched tube voltage, tube current, and exposure time distributions. The technical effect of training with physics-based polychromatic simulation includes improved generalization to real fluoroscopic image characteristics such as beam hardening, scatter, and detector-response nonlinearity, thereby enabling the trained model to produce accurate volumetric reconstructions from clinical fluoroscopic input without requiring rotational acquisition or preoperative CT data.

In some embodiments, the fluoroscopic image acquisition system comprises a fixed bi-plane fluoroscopy system with two orthogonal X-ray sources and detectors acquiring synchronized images simultaneously. The bi-plane configuration provides complementary anatomical information from two orientations within a single acquisition event, thereby supporting volumetric reconstruction in cardiology, neurointerventional, and other clinical domains where continuous translational motion of a single-plane system may be impractical.

In an aspect, the present disclosure provides a computer program product comprising a non-transitory computer-readable medium storing instructions that, when executed by a processor coupled to a graphics processing unit, cause the processor to perform a method. The method comprises receiving, from a fluoroscopic image acquisition system comprising a radiation source and a radiographic image detector, a sequence of N fluoroscopic frames, wherein N is greater than (>) 4, captured during one or more continuous translational sweeps along an extended anatomical region of a patient, the fluoroscopic frames in any image format including native digital or digitized analog video, together with six-degree-of-freedom positional data generated by a spatial tracking system tracking a first fiducial marker mounted to the fluoroscopic image acquisition system and a second fiducial marker affixed to the patient proximate the extended anatomical region for at least one reference frame. The method further comprises producing, from the N fluoroscopic frames, one or more two-dimensional representations, each being a raw fluoroscopic image, a composited panoramic image, or learned feature maps from a trained two-dimensional deep learning network, and back-projecting the two-dimensional representations into one or more three-dimensional voxel grids using projection matrices derived from the positional data. The method further comprises producing a volumetric output by processing the back-projected representations through a trained three-dimensional deep learning network, the volumetric output spanning a spatial extent, in at least one dimension, exceeding the field of view of any single fluoroscopic frame, and registering the volumetric output to a patient coordinate system using spatial tracking measurements of the first and second fiducial markers, the registration being independent of any preoperative volumetric imaging study of the patient and any manual intraoperative landmark-correspondence step.

In some embodiments, the instructions further cause the processor to, prior to back-projection, composite sequential fluoroscopic frames from each sweep orientation into a panoramic image by determining inter-frame spatial relationships from image content by detecting and matching image features in overlapping regions between adjacent frames, composing a cumulative chain of inter-frame geometric transformations from a single tracked reference pose, and back-projecting the panoramic image into a single extended three-dimensional voxel grid for reconstruction in one pass.

In some embodiments, the fluoroscopic image acquisition system includes a mobile C-arm, a fixed fluoroscopic gantry, a robotic imaging arm, a table-integrated fluoroscopic system, or a fixed bi-plane fluoroscopy system. In some embodiments, the spatial tracking system includes visible-light optical cameras, infrared optical cameras, electromagnetic tracking systems, inertial measurement units, mechanical encoders, or hybrid combinations thereof. In some embodiments, the fluoroscopic image data include native digital detector outputs, DICOM images, digital cine sequences, raw detector readouts, or digitized analog video. In some embodiments, the trained neural network model includes convolutional neural networks, transformers, hybrid CNN-transformer architectures, U-Nets, GAN-based networks, diffusion models, variational autoencoders, coordinate-based volumetric decoders, or combinations thereof. The present disclosure therefore provides a broad yet technically grounded framework for extended-field fluoroscopic volumetric reconstruction across multiple hardware environments, tracking densities, image formats, and neural-network architectures.

The disclosure further provides methods, systems, and non-transitory computer-readable media configured to implement the foregoing functions and additional functions described herein.

Conventional fluoroscopic systems generally provide only localized two-dimensional image information within the detector footprint. Anatomy having substantial longitudinal extent, transverse extent, or both, such as the full spine, long bones, the pelvis, and bilateral anatomy, therefore often requires multiple separate fluoroscopic shots taken at different positions. Such workflows can lead to inter-image inconsistency, inefficient radiation usage, and an absence of coherent three-dimensional volumetric data. The present disclosure addresses these issues by combining sequential fluoroscopic views acquired during one or more translational sweeps with associated geometry information and a trained machine-learning reconstruction framework to generate a CT-like volumetric output spanning an anatomical region larger than the native field of view of any individual fluoroscopic frame. The present disclosure therefore supports extended-field fluoroscopic volumetric reconstruction without requiring rotational CBCT acquisition for the extended region of interest.

The present disclosure includes a broad range of spatial-tracking implementations for determining the relationship between the patient and the fluoroscopic imaging system over the course of a sweep. Optical tracking includes visible-light and infrared tracking systems that observe fiducial markers or coded targets. Electromagnetic tracking includes systems in which sensors report six-degree-of-freedom pose relative to a generated field. Inertial tracking includes accelerometers and gyroscopes mounted on the fluoroscopic system, the patient reference, or both. Mechanical tracking includes rotary or linear encoders on translational rails, rotational joints, motorized patient tables, or other movable support structures. Hybrid tracking includes combinations of such sensing modalities fused by a Kalman filter, complementary filter, or another sensor-fusion algorithm. The spatial tracking system 114 therefore includes any sensing arrangement capable of producing geometry information sufficient to determine, constrain, or refine frame-specific projection matrices. The spatial tracking system 114 need not provide a directly measured pose for every fluoroscopic frame.

The present disclosure further recognizes that classical filtered back-projection and Feldkamp-Davis-Kress reconstruction techniques rely on angularly diverse rotational projections and therefore do not naturally apply to translational sweep datasets. The present disclosure instead treats the translational sweep as a source of spatially distributed, geometrically related projections and combines explicit geometry with learned anatomical priors, such as statistical relationships among vertebral shape, alignment, continuity, cortical boundaries, disc spaces, canal geometry, or other anatomical structures learned from training data, to infer volumetric structure from those projections. In this way, the present disclosure supports fluoroscopic acquisition geometries that are fundamentally different from circular or isocentric cone-beam trajectories while still producing a volumetric output suitable for navigation, visualization, segmentation, and related intraoperative uses.

Prior approaches to extending the three-dimensional reconstruction volume from radiographic images have included classical CBCT stitching using multiple orbital sub-volume acquisitions registered via optical tracking; long-length tomosynthesis using custom multi-slot collimators on dedicated gantry systems; and continuous-sweep limited-angle fluoroscopy with deep-learning-based device segmentation. None of these prior approaches combines continuous translational sweep acquisition with neural-network-based volumetric reconstruction using learned feature back-projection to produce an extended-field-of-view three-dimensional volume that exceeds the native detector footprint. Furthermore, prior deep-learning-based reconstruction methods have universally relied on angularly diverse projections (rotational, multi-angle, or cone-beam geometry) because the mathematical foundation of tomographic reconstruction depends on angular sampling of the imaged object's Radon transform. The present disclosure departs from this assumption by demonstrating that a translational sweep geometry, in which projections are acquired at nearly parallel angles from sequential positions along the anatomy, can serve as the basis for deep-learning-based volumetric reconstruction when combined with learned back-projection and three-dimensional refinement, with the spatial diversity of source positions along the sweep direction providing complementary information that the trained neural network exploits to infer three-dimensional structure.

The present disclosure improves fluoroscopic imaging technology by enabling a fluoroscopic image acquisition system that ordinarily provides localized two-dimensional projection images to generate a three-dimensional volumetric image spanning an anatomical extent larger than the native field of view of the detector. More particularly, the present disclosure includes one or more continuous translational sweeps, frame-specific or derived projection geometry, geometry-aware back-projection into one or more voxel grids, and trained three-dimensional neural reconstruction to generate a patient-registered volumetric output from fluoroscopic image data that, in the absence of the disclosed processing, would not itself provide a coherent extended-field three-dimensional representation. The present disclosure further improves intraoperative workflow by automatically registering the reconstructed volume to the patient coordinate system via tracked fiducial markers, thereby avoiding reliance on a preoperative CT scan or manual intraoperative registration while still producing a navigation-ready volumetric result.

The technical problem addressed by the present disclosure is that conventional fluoroscopic imaging systems are physically constrained to a single-frame field of view determined by the dimensions of the radiographic image detector, while existing three-dimensional intraoperative imaging systems generally require expensive orbital cone-beam acquisition geometries, separate preoperative CT studies, and manual intraoperative registration steps. The technical solution disclosed herein combines (i) one or more continuous translational sweeps of the fluoroscopic image acquisition system, (ii) frame-specific projection geometry derived from spatial tracking of fiducial markers on both the fluoroscopic image acquisition system and the patient, and (iii) a trained neural reconstruction pipeline that performs geometry-aware back-projection and volumetric refinement. The technical effects of this combination include (a) generation of a CT-like volumetric output spanning an anatomical extent that exceeds the native field of view of any single fluoroscopic frame using standard mobile-C-arm or fixed-fluoroscopy hardware, (b) automatic registration of the volumetric output to the patient coordinate system without dependence on a preoperative volumetric imaging study or on manual intraoperative landmark-correspondence procedures, and (c) cumulative radiation exposure that is materially lower than equivalent-coverage orbital cone-beam acquisitions because the disclosed translational sweep geometry uses approximately 10-100 frames per sweep rather than the 50-1,000 projections characteristic of orbital cone-beam acquisition. The disclosed system therefore enables three-dimensional volumetric imaging in surgical environments where conventional cone-beam-CT systems are unavailable, unaffordable, or unsuitable due to dose, size, or workflow constraints.

### BRIEF DESCRIPTION OF THE DRAWINGS

FIG. 1A is a conceptual block diagram of a surgical navigation and imaging system configured for extended-field volumetric reconstruction, showing a fluoroscopic image acquisition system, a calibration target, fiducial markers, a synchronizing device, a tracking system, a computing system, and a display environment, together with their functional relationships for acquiring fluoroscopic images, determining imaging geometry, reconstructing a volumetric output, and registering the volumetric output to patient space.

FIG. 1B is a conceptual illustration of a representative procedural environment for the surgical navigation and imaging system of FIG. 1A, showing a patient on a surgical table, a fluoroscopic image acquisition system performing a translational sweep along an anatomy of interest, one or more external cameras positioned to track fiducial markers on the patient and the fluoroscopic image acquisition system, and associated hardware used to acquire fluoroscopic images and tracked positional data for extended-field volumetric reconstruction and patient-space registration.

FIG. 2 illustrates sequential fluoroscopic frames captured during a continuous anterior-posterior sweep, including overlapping frames and extended anatomical coverage beyond the native field of view of the fluoroscopic detector.

FIG. 3 illustrates a lateral sweep sequence showing a second set of fluoroscopic images captured during translational motion, including overlapping frame coverage of the anatomy of interest.

FIG. 4 is a block diagram of a general reconstruction framework including a two-dimensional representation stage, a geometry-aware back-projection stage, a multi-frame aggregation stage, and a three-dimensional volume refinement stage configured to produce a volumetric reconstruction and, in some aspects, one or more segmentation outputs.

FIG. 5 is a block diagram of a first reconstruction architecture in which a shared two-dimensional encoder extracts multi-resolution feature maps from fluoroscopic frames, the feature maps are selectively back-projected at multiple three-dimensional scales, aggregated across frames, and processed by a decoder-only three-dimensional network.

FIG. 6 is a block diagram of a second reconstruction architecture in which per-view two-dimensional encoders extract features from different fluoroscopic view streams, the features are mixed across levels and views, back-projected into multi-scale volumetric cubes, and processed by a full three-dimensional encoder-decoder network.

FIG. 7 is a block diagram of a third reconstruction architecture in which raw fluoroscopic images are back-projected directly into a volumetric tensor and processed by a monolithic three-dimensional encoder-decoder network without a learned two-dimensional encoder.

FIG. 8 illustrates a geometry-aware differentiable back-projection arrangement showing how a two-dimensional feature map is projected into a three-dimensional voxel grid using a projection matrix and a grid-sampling operation.

FIG. 9 illustrates a multi-scale back-projection pyramid showing multiple resolution levels of back-projected cubes and a configurable feature-to-scale mapping between two-dimensional encoder levels and three-dimensional volumetric scales.

FIG. 10A illustrates extended field-of-view reconstruction via sub-volume tiling, including multiple reconstructed sub-volumes, overlap zones, and weighted blending for generation of a unified extended reconstructed volume.

FIG. 10B illustrates tracker-based panoramic stitching in which sequential fluoroscopic frames from one or more sweep orientations are composited into one or more panoramic images using known or derived per-frame spatial offsets, and the panoramic images are back-projected into a single extended three-dimensional voxel grid for volumetric reconstruction.

FIG. 10C illustrates image-based panoramic stitching in which inter-frame spatial relationships are determined from image content by detecting and matching features in overlapping regions between adjacent frames, composing a cumulative transform chain from a tracked reference frame, and generating a panoramic image registered to the patient coordinate system without requiring per-frame spatial tracking.

FIG. 11 is a sweep acquisition timing diagram showing translational motion of the imaging system, sequential frame positions or acquisition events, associated tracking or pose capture timing, and X-ray pulse synchronization over one or more sweep intervals.

FIG. 12 illustrates a training pipeline in which CT volumes are paired with simulated digitally reconstructed radiographs generated by a polychromatic X-ray simulator using clinically matched acquisition parameters and projection geometry for training of a neural reconstruction model.

FIG. 13 illustrates an automatic registration pipeline in which fiducial tracking and associated geometry information place a reconstructed volumetric output into a patient coordinate system for immediate surgical navigation or guidance.

FIG. 14 illustrates a multi-head output architecture in which a shared three-dimensional feature volume branches into multiple output heads including a CT-like reconstruction head, a tissue segmentation head, a metal segmentation head, and an anatomical-part segmentation head.

FIG. 15 illustrates a per-frame inline calibration workflow in which a raw fluoroscopic frame containing visible radiopaque calibration features undergoes marker detection, feature extraction, point-correspondence formation, perspective-n-point pose estimation, optional refinement, and artifact removal to produce a calibrated fluoroscopic frame with associated imaging geometry.

FIG. 16 illustrates representative calibration-device embodiments including wire-based, spherical-marker, coded-marker, and grid-or plate-based calibration structures configured to provide two-dimensional-to-three-dimensional point correspondences for frame-specific calibration.

FIG. 17 illustrates a pipeline variant taxonomy showing representative reconstruction configurations including per-frame and panoramic-first paths, learned and raw-image two-dimensional representations, and decoder-only or full encoder-decoder three-dimensional processing.

FIG. 18 illustrates representative tracking-density modes including per-frame tracking, sparse tracking with interpolation or extrapolation, and single-reference tracking in which a tracked reference frame is combined with image-based inter-frame registration to derive remaining frame geometries.

FIG. 19 illustrates a transverse translational sweep acquisition for wide-anatomy imaging in which the fluoroscopic imaging system translates across an anatomy such as a pelvis or bilateral anatomy while acquiring sequential overlapping frames that support reconstruction of a volume whose transverse extent exceeds the detector's field of view.

### DETAILED DESCRIPTION

The present disclosure will be more completely understood through the following description, which should be read in conjunction with the drawings. In this description, like reference characters identify like or analogous elements throughout the several views except where the context clearly indicates otherwise. The systems, methods, and computer-implemented processes described herein are representative and are provided so that a person of ordinary skill in the art can make and use the present disclosure. Variations in hardware, software, acquisition geometry, calibration technique, reconstruction pipeline, neural-network architecture, and clinical workflow remain within the scope of the present disclosure so long as the disclosed functionality is preserved. The incorporated patents and applications provide additional environmental and implementation support for the fluoroscopic, calibration, reconstruction, and registration frameworks referenced herein.

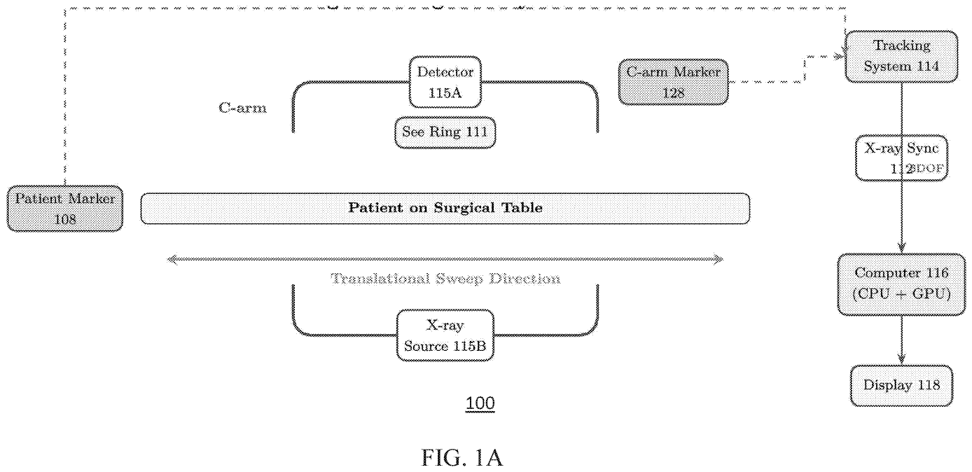

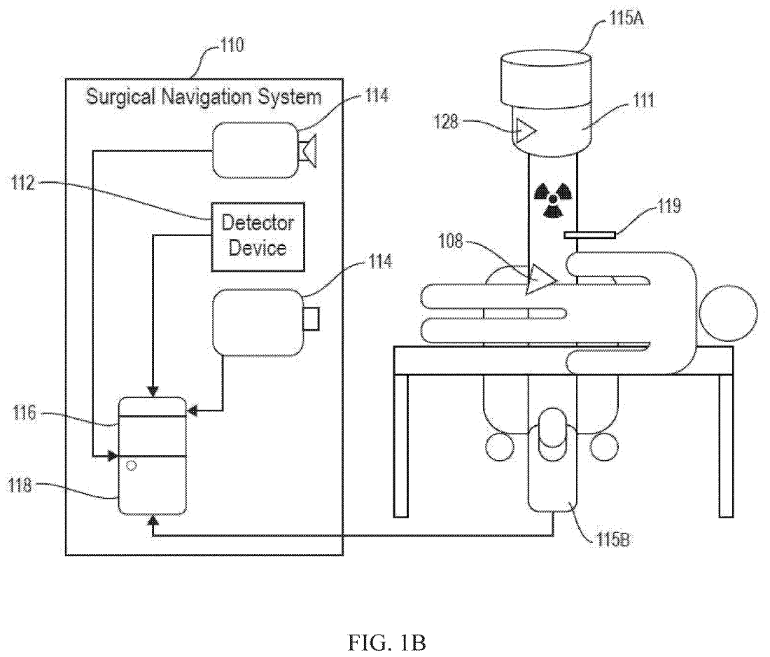

Referring to FIG. 1A, a surgical navigation and imaging system 110 includes a fluoroscopic image acquisition system having a radiation source 115B and a radiographic image detector 115A, which together define an imaging assembly that acquires radiographic image data relative to a patient. The system 110 further includes a calibration target 111, a synchronizing device or radiation detector 112, one or more tracking devices 114, a computer 116, a display interface 118, a patient reference marker 108, and an imaging-system reference marker 128. In some implementations (i.e., embodiments), the system 110 further includes one or more tracked instruments 119 that are interpreted relative to the patient and to the reconstructed volumetric data. The patient reference marker 108 is fixed relative to the patient proximate to the anatomy to be reconstructed, and the imaging-system reference marker 128 is fixed relative to the fluoroscopic imaging system, such as on or near the calibration target 111 or the detector housing. The tracking devices 114 determine the spatial relationship of the markers 108 and 128 in a common coordinate space and provide corresponding information to the computer 116. The synchronizing device 112 associates radiographic acquisition timing with tracking timing, image timing, or both, so that image data acquired by the detector 115A can be paired with geometry data corresponding to the state of the fluoroscopic imaging system and the patient at the time of acquisition. The computer 116 processes fluoroscopic image data, geometry data, or both, to generate a volumetric reconstruction, one or more segmentation outputs, one or more registration outputs, or combinations thereof, and the display interface 118 presents such outputs to a user. This overall environment is also consistent with the tracking, calibration, registration, and fluoroscopic-navigation context described in the incorporated family materials.

The fluoroscopic image acquisition system that includes the source 115B and the detector 115A also includes a mobile C-arm, a fixed fluoroscopy gantry, a robotic imaging arm, a table-integrated fluoroscopic system, a bi-plane fluoroscopy system, or another radiographic imaging arrangement capable of acquiring sequential projection images of anatomy. The calibration target 111 includes a radiopaque reference body having known geometry, such as a wire-based calibration ring, a spherical-marker frame, a coded fiducial structure, a grid or plate carrying radiopaque features, or other geometry-bearing reference. The synchronizing device 112 includes a radiation detector, trigger interface, control board, timing controller, pulse monitor, timestamping device, or integrated acquisition controller that relates radiographic events to pose events or image events. The tracking devices 114 include visible-light optical trackers, infrared trackers, electromagnetic trackers, inertial sensing units, mechanical encoders, or fused multi-sensor arrangements. The computer 116 includes one or more processors, graphics processing units, memory devices, and storage devices configured to execute the calibration, compositing, back-projection, reconstruction, registration, segmentation, and display functions described herein. The display interface 118 includes a monitor, workstation display, navigation display, touchscreen, augmented-reality interface, or another interface configured to present volumetric or guidance data. The tracked instrument 119 includes a surgical tool, pointer, drill, probe, implant inserter, robotic end effector, or other tracked article. The patient reference marker 108 and imaging-system reference marker 128 each include a fiducial body, coded marker, retroreflective marker, active optical marker, electromagnetic sensor, inertial-referenced structure, or another trackable reference having a stable relationship to the respective patient or imaging system.

The surgical navigation system 110 includes a local or distributed computing arrangement in which the computer 116 executes image acquisition coordination, calibration, reconstruction, registration, segmentation, and display logic either within a single workstation or across multiple coupled processors. The tracking devices 114 continuously observe the patient reference marker 108 and imaging-system reference marker 128 during fluoroscopic acquisition, and the synchronizing device 112 detects radiographic pulse events from the source 115B and causes corresponding image timing information, pose timing information, or both, to be associated with the frame acquired by detector 115A. In this way, the system 110 maintains a temporally coherent relationship between the fluoroscopic image sequence and the patient-relative imaging geometry throughout the sweep. Related fluoroscopic-navigation and coordinate-management environments are also described in the incorporated registration-transform and biplanar-reconstruction family materials, and those disclosures provide additional support for patient-space alignment, detector and source geometry, and navigation integration in the operating-room setting.

The system 110 is therefore a particular fluoroscopic imaging and navigation machine in which the source 115B, detector 115A, synchronizing device 112, tracking devices 114, fiducial markers 108 and 128, and computer 116 cooperate to generate machine-derived inputs for volumetric reconstruction. In operation, the detector 115A acquires fluoroscopic frames, the synchronizing device 112 associates those frames with acquisition timing information, the tracking devices 114 determine tracked or derivable spatial relationships involving the fiducial markers 108 and 128, and the computer 116 uses those machine-generated inputs to determine projection matrices and reconstruct a patient-registered volumetric image. In representative implementations, the operating-room and navigation environment corresponds generally to the fluoroscopic-navigation environments described in incorporated U.S. Pat. No. 12,444,127 and the incorporated registration-transform family materials, including use of tracked patient and imaging-system references, calibrated source and detector geometry, and navigation displays for patient-referenced volumetric information. The incorporated family materials further describe representative surgical-navigation environments in which tracked instruments are aligned with a reconstructed three-dimensional volume through a shared registration transform, and those disclosures provide additional support for the use of the tracked instrument 119, the fiducial markers 108 and 128, and the display interface 118 in conjunction with the reconstructed volumetric output of the present disclosure.

Referring to FIG. 1B, a surgical navigation and imaging system 110 is shown in a representative procedural environment. A patient is positioned on a surgical table, and a fluoroscopic image acquisition system, such as a C-arm, is arranged relative to the patient with a radiation source 115B disposed on one side of the patient and a radiographic image detector 115A disposed on the opposite side of the patient. In the illustrated example, the fluoroscopic image acquisition system performs an anterior-posterior sweep along the patient's spine so that sequential fluoroscopic frames are acquired at different translational positions relative to the anatomy of interest. One or more external cameras 114 are positioned proximate the surgical table and track a patient reference marker 108 attached to the patient and an imaging-system reference marker 128 attached to the calibration target 111 or another portion of the fluoroscopic image acquisition system. A synchronizing device 112 detects radiographic acquisition events and associates image-acquisition timing with corresponding pose information, and a computer 116 receives the fluoroscopic image data and tracked positional data for further processing. A display interface 118 presents reconstructed volumetric information, navigation information, or both, and one or more tracked surgical instruments 119 may be registered relative to the reconstructed volume and the patient coordinate system.

FIG. 1B therefore illustrates the physical operating-room arrangement in which the components of system 110 cooperate during fluoroscopic acquisition. The external cameras 114 may include visible-light optical cameras, infrared tracking cameras, or other optical tracking devices capable of determining real-time six-degree-of-freedom pose information for the reference markers 108 and 128. In representative implementations, the synchronizing device 112 identifies X-ray emissions relative to a threshold level and causes corresponding pose information for the patient and fluoroscopic imaging system to be captured or associated with the acquired fluoroscopic frame. The fluoroscopic image acquisition system may be a mobile C-arm, a fixed fluoroscopy system, a robotic imaging arm, or another radiographic imaging arrangement capable of acquiring sequential fluoroscopic images during translational sweep motion. The components of the surgical navigation and imaging system 110 may be integrated in a common housing or distributed across multiple interoperable devices coupled through suitable wired or wireless network infrastructure.

The patient reference marker 108 and imaging-system reference marker 128 serve as fiducial references for determining the spatial relationship between the patient and the fluoroscopic image acquisition system during the sweep. In representative implementations, the patient reference marker 108 is secured to the patient proximate the anatomical region to be reconstructed, and the imaging-system reference marker 128 is secured to the calibration target 111, detector housing, or another portion of the fluoroscopic image acquisition system that maintains a known spatial relationship to the radiation source 115B and radiographic image detector 115A. The resulting tracked geometry allows the system 110 to determine or constrain frame-specific projection matrices for the sequential fluoroscopic frames acquired during the sweep, thereby supporting the geometry-aware back-projection and volumetric reconstruction operations described elsewhere herein.

FIG. 1B complements FIG. 1A by illustrating a representative hardware and procedural arrangement for the disclosed system, whereas FIG. 1A illustrates a more abstract system architecture and functional relationship among components. Representative fiducial-marker, reconstruction, and registration environments suitable for use with the system of FIG. 1B are described further in the incorporated related patents and applications, including the incorporated registration-transform, reconstruction, calibration, and marker-family disclosures.

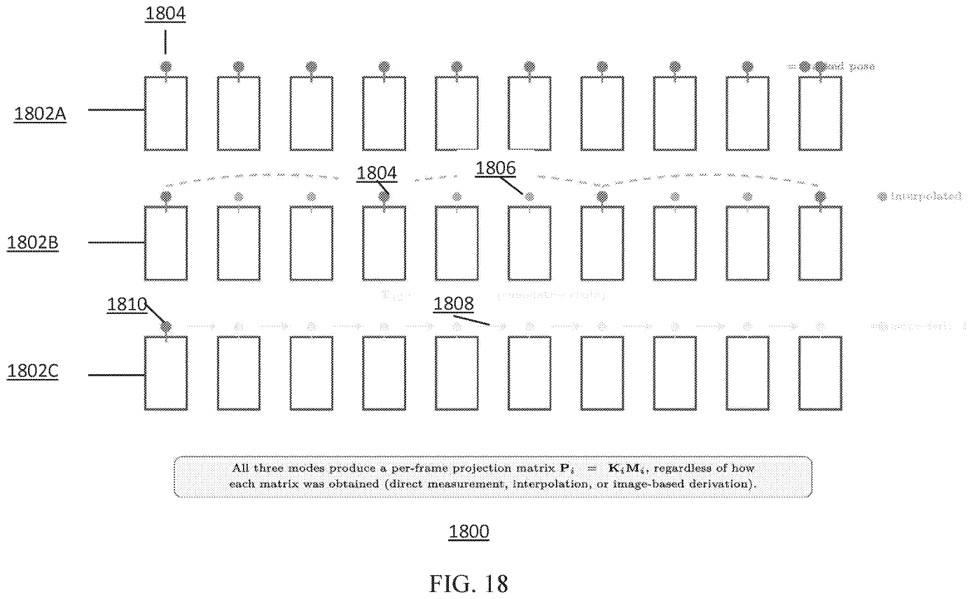

Referring to FIG. 18, a tracking-density-mode diagram 1800 includes a per-frame tracking mode 1802A, a sparse tracking mode 1802B, and a single-reference tracking mode 1802C. In the per-frame tracking mode 1802A, directly measured poses 1804 are obtained for every frame. In the sparse tracking mode 1802B, directly measured poses 1804 are available only for selected frames, and interpolated poses 1806 are derived for intermediate frames. In the single-reference tracking mode 1802C, only a reference pose 1810 is directly measured; remaining poses are obtained as image-derived poses 1808 from inter-frame registration. In all cases, the system produces a projection matrix for each frame in the fluoroscopic sequence, regardless of whether the projection matrix is obtained through direct tracking measurement, interpolation between tracked frames, image-based inter-frame registration, or any combination thereof. The downstream geometry-aware back-projection and reconstruction logic operates on the resulting projection matrices according to their geometry rather than according to the manner in which they were obtained.

In example implementations, the directly measured poses 1804 include optical-tracker outputs, electromagnetic outputs, encoder-derived poses, inertial-assisted poses, or fused-sensor poses. The interpolated poses 1806 include linear interpolation, spline interpolation, model-based interpolation, smoothed interpolation, extrapolation, or otherwise inferred geometry states between directly measured points. The image-derived poses 1808 include transform-chain-derived poses, feature-registration-derived poses, homography-derived poses, or otherwise image-inferred geometry. The reference pose 1810 includes a first-frame pose, an anchor-frame pose, a checkpoint pose, or another directly measured pose used to ground the frame sequence in a physical coordinate system. This range of tracking densities improves deployability across different hardware and workflow constraints while preserving consistent downstream processing.

In embodiments that employ sparse tracking or only a single tracked reference pose, the present disclosure includes derivation of frame-to-frame spatial relationships from image content. The image-based registration pipeline computes inter-frame transforms by detecting and matching features in overlapping image regions and composing the resulting inter-frame geometric transformations with the single known reference pose. Suitable feature detection methods include classical keypoint detectors such as SIFT, ORB, SURF, or Harris corner detection; frequency-domain methods such as phase cross-correlation; learned feature extractors that generate dense or sparse descriptors optimized for fluoroscopic image content; and radiopaque calibration-device features that provide engineered high-contrast landmarks visible in each frame. Suitable natural anatomical features include vertebral endplates, pedicle screw heads, rib-vertebra intersections, cortical bone edges, trabecular texture, and implanted-device features. Feature correspondences between adjacent frames are established using nearest-neighbor matching, including k-nearest-neighbors matching with Lowe's ratio test, learned matching networks, or other matching techniques, and outliers are rejected using RANSAC or another robust estimator. The resulting inter-frame transform may be a translation, Euclidean transform, similarity transform, affine transform, homography, or learned warp, depending on the image content and sweep geometry. The system then composes the inter-frame transforms into a cumulative transform chain and may further mitigate drift using bundle adjustment, loop closure, sparse tracking checkpoints, bidirectional chaining, or other drift-control strategies. The present disclosure therefore includes geometry inference from fluoroscopic image content itself, not only from external tracking hardware.

Unlike prior systems that acquire a small number of static fluoroscopic images at a single anatomical location, the present disclosure includes fluoroscopic acquisition during one or more continuous translational sweeps along a sweep axis determined by the anatomy of interest. For spinal and long-bone applications, the sweep axis is typically aligned with the patient's longitudinal axis. For wide anatomies such as the pelvis, head, shoulders, or bilateral extremities, the sweep axis is typically aligned transversely, anterior-posteriorly, obliquely, or along another direction selected to extend coverage beyond the detector footprint. The sweep can be linear, curvilinear, piecewise linear, segmented, manually guided, robotically controlled, or otherwise positionally varied so long as sequential frames are acquired at different positions relative to the anatomy.

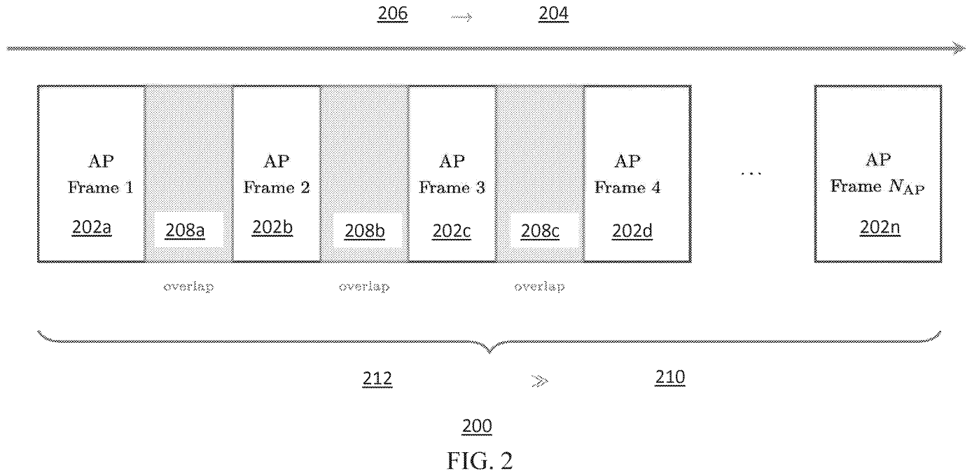

Referring to FIG. 2, an anterior-posterior sweep sequence 200 includes a plurality of sequential AP fluoroscopic frames 202a, 202b, 202c, 202d, 202n acquired during translational movement of the fluoroscopic imaging system along a sweep direction 204 relative to an anatomy of interest 206. Adjacent frames partially overlap in respective overlap regions 208a, 208b, 208c. Each individual frame is bounded by the native detector field of view 210, while the aggregate set of frames spans an extended anatomical coverage 212 that exceeds the native detector field of view 210 of any one frame. The AP frames 202a, 202b, 202c, 202d, 202n are acquired at different translational positions and later associated with frame-specific or derived projection geometry for reconstruction. The overlap regions 208a, 208b, 208c support registration, blending, or feature continuity between neighboring frames. The anatomy of interest 206 includes the spine, a long bone, the pelvis, a multi-joint segment, bilateral anatomy, or another anatomical region whose extent exceeds a single detector footprint.

In exemplary workflows, the AP fluoroscopic frames 202a, 202b, 202c, 202d, 202n are acquired while the operator or a motorized transport mechanism translates the fluoroscopic imaging system at a controlled speed during active fluoroscopy, such that each frame corresponds to a different translational position along the sweep direction 204. Each frame is later associated with directly measured or derived geometry, thereby allowing the overlap regions 208a, 208b, 208c to serve not only as visual overlap but also as sources of registration continuity, blending continuity, and multi-frame anatomical correspondence. The AP sweep sequence 200 therefore supports both direct frame-wise reconstruction workflows and panoramic or tiled workflows in which neighboring frames contribute jointly to a larger volumetric representation.

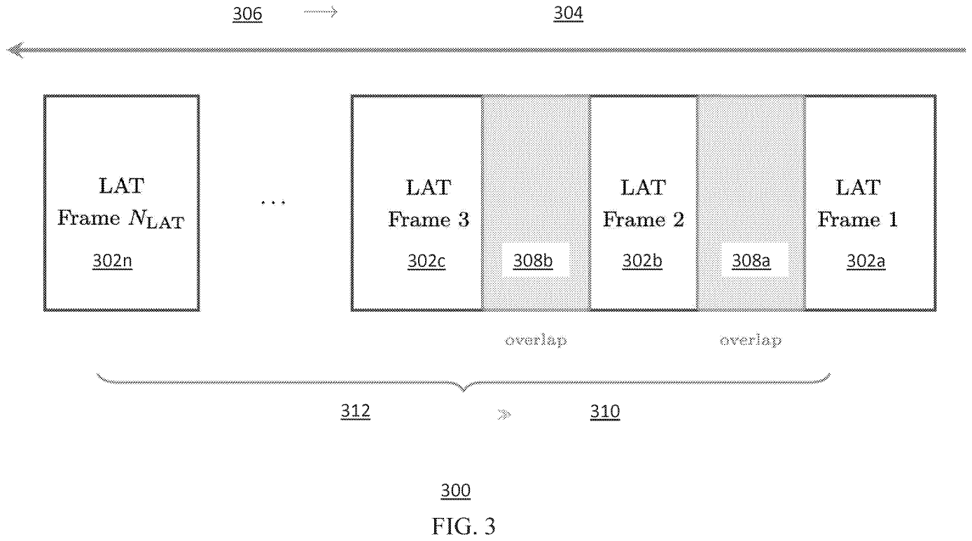

Referring to FIG. 3, a lateral sweep sequence 300 includes a plurality of sequential lateral fluoroscopic frames 302a, 302b, 302c, 302n acquired during translational motion along a sweep direction 304 relative to an anatomy of interest 306. Adjacent frames partially overlap in overlap regions 308a, 308b. Each lateral frame is bounded by a native detector field of view 310, while the sequence as a whole spans an extended anatomical coverage 312. The lateral frames 302a, 302b, 302c, 302n provide complementary spatial information from a second orientation and are processed in conjunction with the AP sweep sequence 200 to support volumetric inference over anatomy larger than the detector footprint. The AP and lateral sweeps can proceed in the same direction, in opposite directions, or in another paired sequence selected to improve workflow efficiency and anatomical coverage. Additional oblique or otherwise oriented sweeps may also included.

In exemplary workflows, the lateral fluoroscopic frames 302a, 302b, 302c, 302n are acquired while the operator or a motorized transport mechanism translates the fluoroscopic imaging system at a controlled speed during active fluoroscopy, such that each frame corresponds to a different translational position along the sweep direction 304. In some implementations, the lateral sweep proceeds in an opposite translational direction from the AP sweep so that overall workflow time is reduced. Each lateral frame is associated with directly measured or derived geometry, and the overlap regions 308a, 308b therefore support not only visual continuity but also registration continuity, blending continuity, and multi-frame anatomical correspondence.

The overlap fraction between neighboring frames can be selected as a function of detector size, anatomy size, sweep speed, frame interval, and target reconstruction quality. In one representative expression, the overlap fraction α is defined as α=1−v·Δt/D, where v is translational speed, Δt is inter-frame time, and D is the detector field of view along the sweep direction. Greater overlap improves continuity and registration robustness, while lower overlap reduces the number of exposures while preserving enough shared content for fusion. The total number of frames N can be greater than four and often falls within a range such as about 10 to about 100 or more depending on the anatomy and desired density of sampling.

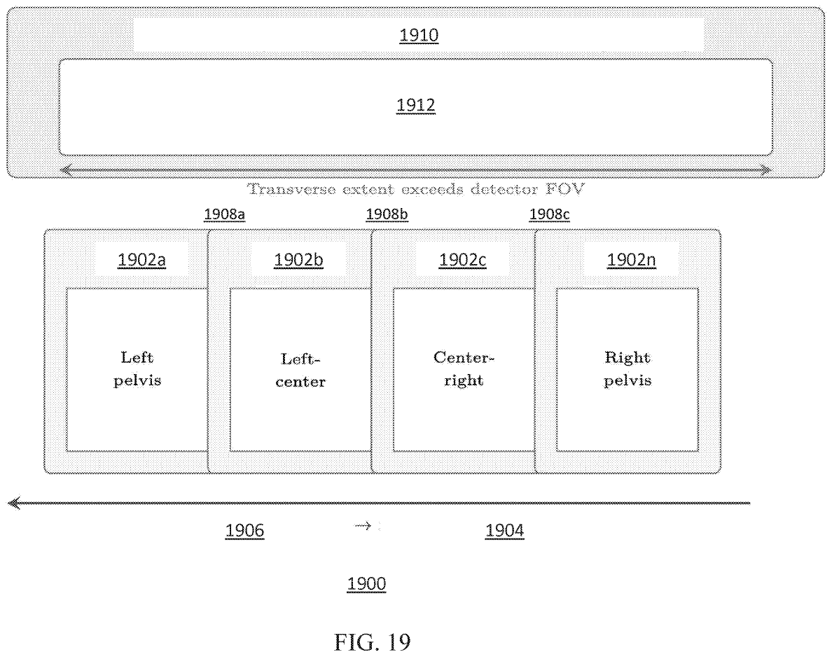

Referring to FIG. 19, a transverse sweep acquisition arrangement 1900 includes sequential transverse fluoroscopic frames 1902a, 1902b, 1902c, 1902n acquired during motion along a transverse sweep direction 1904 relative to a wide anatomy 1906, such as the pelvis. Adjacent frames overlap in overlap regions 1908a, 1908b, 1908c, each frame is bounded by the native detector field of view 1910, and the sequence spans an extended transverse coverage 1912 exceeding that detector field of view 1910. The same calibration, geometry-aware back-projection, neural reconstruction, and, where applicable, tiling or blending pipeline applies to the transverse sweep, except that the extended-grid dimension is oriented transversely, such as along an x-axis, rather than longitudinally along a z-axis. The present disclosure therefore includes extended-field reconstruction not only along a cranial-caudal dimension but also across wide anatomy in a transverse or otherwise in-plane direction.

In example implementations, the wide anatomy 1906 includes the pelvis, bilateral hips, the head, the shoulders, bilateral knees, bilateral feet, or another anatomical target which lateral or in-plane extent exceeds the native detector field of view 1910. In some workflows, a transverse sweep along direction 1904 is combined with an anterior-posterior in-plane sweep or another in-plane sweep so that the resulting reconstructed volume exceeds the detector footprint in two in-plane dimensions. The same calibration, geometry-aware back-projection, neural reconstruction, tiling, blending, or panoramic compositing framework described elsewhere herein also applies to such multi-axis wide-anatomy acquisition.

The fluoroscopic frames are not limited to a single image format. The present disclosure includes native digital detector outputs, DICOM images, digital cine sequences, proprietary or standard cine digital formats, raw detector readouts, TIFF, PNG, NIfTI, and other high-bit-depth or lower-bit-depth digital formats, as well as analog-video-derived image streams such as digitized s-video, composite video, or component video. High-bit-depth digital acquisition preserves detector dynamic range for soft-tissue contrast, metal characterization, and low-contrast detectability. Analog-derived acquisition remains within the present disclosure even though such images can include reduced bit depth, analog noise, interlacing artifacts, gamma nonlinearity, and cable-induced signal degradation. The reconstruction pipeline therefore includes a broad family of fluoroscopic image representations suitable for geometry-aware machine-learning reconstruction, including any image format including native digital formats or digitized analog video.

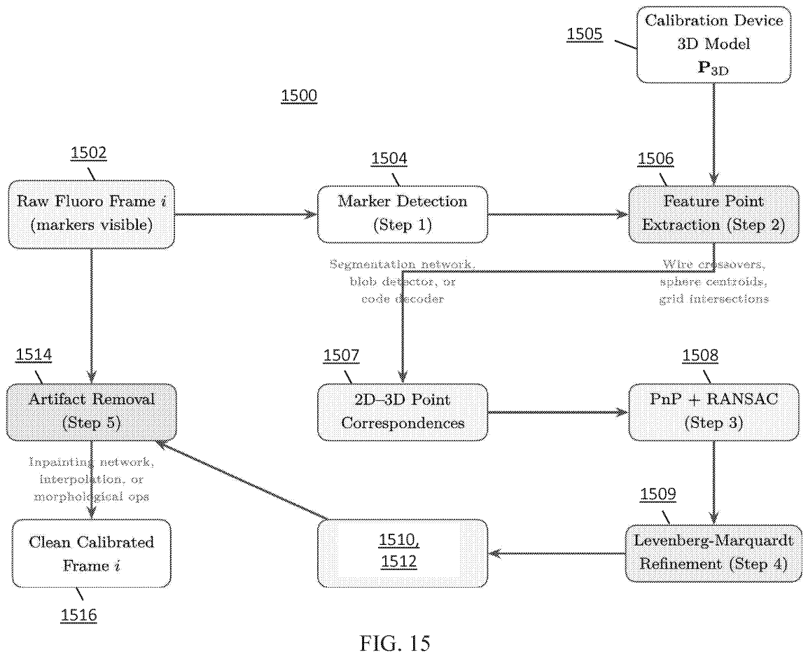

Referring to FIG. 15, an inline calibration workflow 1500 receives a raw fluoroscopic frame 1502 containing a visible calibration device 1505. A marker-detection block 1504 identifies visible features of the calibration device 1505 in the fluoroscopic frame 1502. A feature-point extraction block 1506 determines corresponding two-dimensional feature locations for the detected features. The workflow then matches the two-dimensional feature locations to known three-dimensional feature positions of the calibration device 1505 to establish point correspondences 1507. A PnP pose-estimation block 1508 computes imaging geometry from the point correspondences 1507, and a Levenberg-Marquardt refinement block 1509 refines the computed geometry. Intrinsic and extrinsic matrix outputs 1510 and 1512 are then produced, and an artifact-removal block 1514 generates a calibrated fluoroscopic frame 1516. Because the calibration device 1505 remains in the field during acquisition, the workflow 1500 supports per-frame or repeatedly refreshed calibration without requiring a separate phantom session or static pre-imaging calibration procedure. Each fluoroscopic frame therefore contains a radiopaque calibration device comprising a plurality of radiopaque markers at known three-dimensional coordinates, and per-frame intrinsic and extrinsic camera parameters are computed from detected marker features by establishing two-dimensional-to-three-dimensional point correspondences and solving the perspective-n-point problem.

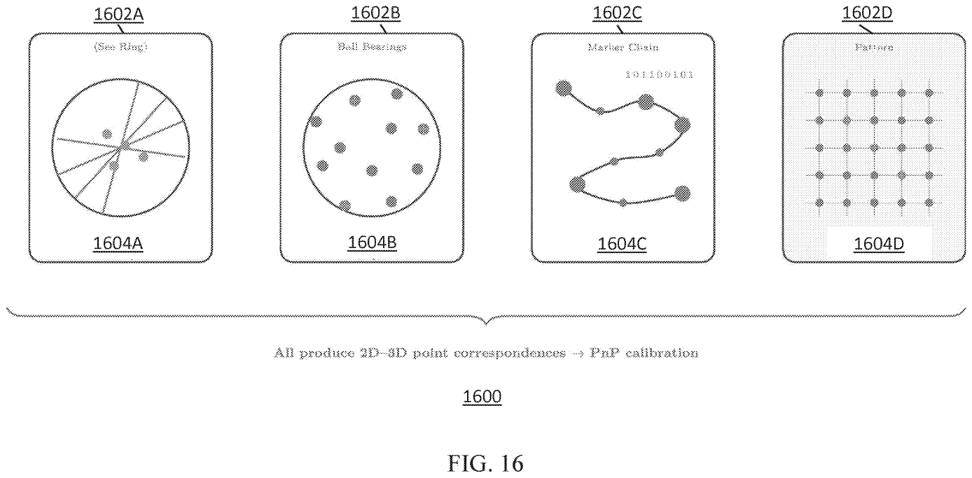

Referring to FIG. 16, calibration device embodiments 1600 include the wire-based calibration device 1602A with wire crossover features 1604A, the spherical-marker calibration device 1602B with sphere centroids 1604B, the coded-marker calibration device 1602C with coded marker pattern 1604C, and the grid or plate calibration device 1602D with grid intersections 1604D. The calibration device 1505 may assume any geometry that produces identifiable features in a radiographic projection and has known three-dimensional coordinates. Suitable examples include linear markers such as radiopaque wires traversing an interior passage at different known depths; spherical markers such as ball bearings distributed in a surveyed helical, grid-like, or otherwise known three-dimensional arrangement; bead, pellet, or cylindrical markers embedded in or attached to a support structure; coded marker arrangements using different sizes or shapes in a non-repeating sequence; planar or three-dimensional grids, plates, or mesh patterns; and hybrid combinations of the foregoing. In one representative implementation, the wire-based calibration device 1602A corresponds generally to the wire-based calibration structures described in incorporated U.S. Pat. No. 12,462,469, entitled “Wire-Based Calibration Apparatus for X-ray Imaging Systems,” issued Nov. 4, 2025, including radiopaque wire arrangements configured to provide projected wire features usable for fluoroscopic calibration. The spherical, coded, grid, and other geometries remain within the present disclosure as alternative calibration structures.

In one representative sequence, the inline calibration workflow 1500 proceeds through visible marker detection at block 1504, extraction of identifiable image features at block 1506, formation of two-dimensional-to-three-dimensional point correspondences 1507, initial PnP-based pose estimation at block 1508, nonlinear refinement at block 1509, construction of the intrinsic and extrinsic matrix outputs 1510, 1512, optional integration of the calibrated frame geometry with tracked patient and imaging-system markers so that frame geometry is consistent across the sweep, and artifact removal at block 1514. In wire-based implementations, the marker-detection block 1504 can include segmentation of individual wires and identification of wire crossovers or gap features. In spherical-marker implementations, block 1504 can include blob or centroid detection. In coded-marker implementations, block 1504 can further include decoding of marker identity from size or shape patterns. The resulting calibrated fluoroscopic frame 1516 is therefore both geometrically characterized and visually prepared for downstream reconstruction.

Regardless of marker geometry, the mathematical core of the calibration is geometry-agnostic and operates on two-dimensional-to-three-dimensional point correspondences. In one implementation, the perspective-n-point problem is solved using RANSAC with the SQPNP algorithm to obtain initial rotation and translation estimates, after which the pose is refined using Levenberg-Marquardt optimization on the inlier set. When the spatial tracking system 114 provides tracked poses of both the patient marker 108 and the imaging-system marker 128, a transform manager integrates the resulting calibration with the tracked fiducial geometry so that all per-frame calibration results are spatially consistent across the sweep. This per-frame inline calibration eliminates the need for any separate calibration step, phantom acquisition, or pre-imaging session and is especially useful in the sweep-acquisition mode where the fluoroscopic imaging system is continuously moving and its geometry changes from frame to frame.

After calibration, calibration-device artifacts are digitally removed from each frame, producing clean images for input to the reconstruction pipeline. Artifact removal may be performed by any suitable method, including a trained inpainting network, classical interpolation-based inpainting, patch-based or exemplar-based filling, median filtering, morphological operations, or any hybrid or future artifact-removal method. Thus, radiopaque calibration device artifacts may be digitally removed from each frame prior to computation of the two-dimensional representation.

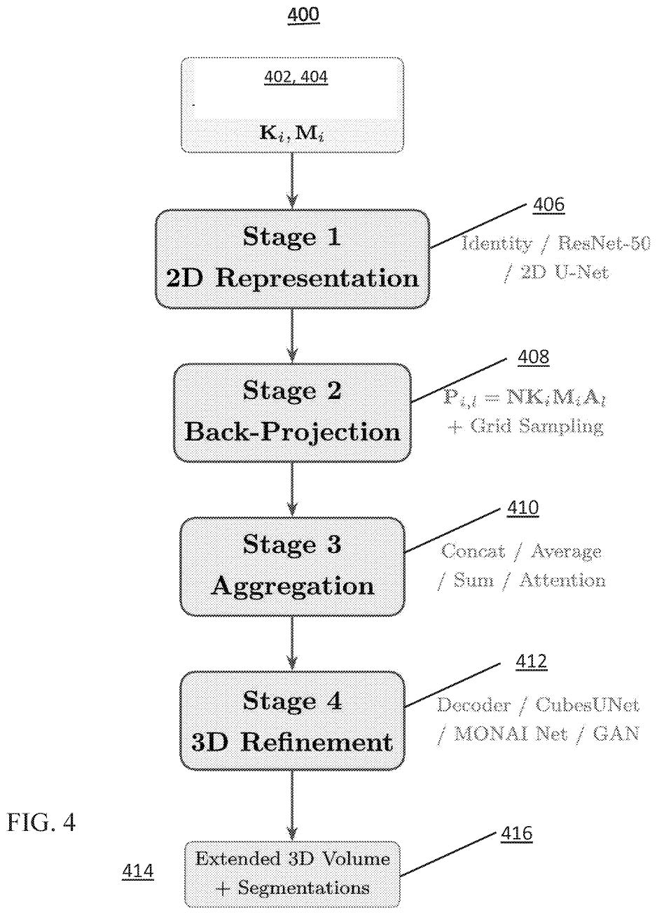

Referring to FIG. 4, a reconstruction framework 400 receives fluoroscopic frames 402 and associated spatial metadata 404. The reconstruction framework 400 includes a two-dimensional representation stage 406, a geometry-aware back-projection stage 408, a multi-frame aggregation stage 410, and a three-dimensional volume refinement stage 412. The reconstruction framework 400 generates a volumetric output 414 and, in some embodiments, one or more segmentation outputs 416. The framework 400 is configured to produce, from the fluoroscopic frames 402, one or more two-dimensional representations; back-project the one or more two-dimensional representations into one or more three-dimensional voxel grids using projection matrices derived from the spatial metadata 404; and produce a volumetric output by processing the back-projected representations through a trained three-dimensional deep learning network.

The two-dimensional representation stage 406 includes an identity representation in which the fluoroscopic image itself is used directly, and also includes learned feature extraction using convolutional neural networks, vision transformers, hybrid CNN-transformer architectures, U-Net-style encoders, multi-layer perceptron mixers, Fourier neural operators, and other two-dimensional feature extractors. Per-view encoders can be shared across views or independently parameterized for AP, lateral, or other acquisition orientations. The two-dimensional representation can be computed from individual fluoroscopic frames or from one or more panoramic composites formed by spatially registering and blending sequential frames. Accordingly, each two-dimensional representation may be selected from a raw fluoroscopic image, a composited panoramic image formed by spatially registering and blending a plurality of sequential frames, or learned feature maps extracted by a trained two-dimensional deep learning network operating on individual fluoroscopic frames or on a composited panoramic image.

The reconstruction framework 400 is not limited to a single fixed ordering of the functional stages 406, 408, 410, 412. The present disclosure includes per-frame ordering in which individual frames are first transformed into two-dimensional representations and then back-projected, panoramic-first ordering in which multiple sequential frames are composited before two-dimensional encoding or back-projection, and raw-image ordering in which the fluoroscopic images are projected into volumetric space without a learned two-dimensional encoder. The reconstruction framework 400 therefore defines a machine-implemented imaging pipeline that receives fluoroscopic image data and associated spatial metadata from the fluoroscopic image acquisition system, produces one or more two-dimensional representations, back-projects those representations into one or more three-dimensional voxel grids using projection matrices derived from the positional data, and generates a volumetric output spanning a spatial extent, in at least one dimension, exceeding the field of view of any single fluoroscopic frame.

The multi-frame aggregation stage 410 combines back-projected information from multiple frames, multiple views, or both into one or more volumetric tensors. The aggregation includes channel concatenation, averaging, summation, learned attention, confidence-weighted fusion, recurrent accumulation, graph-based integration, or other operation preserving or compressing view-dependent information. In some implementations, one cube is produced per frame per pyramid level and then aggregated across frames. In other implementations, raw-frame channels are directly accumulated into a shared tensor. The present disclosure includes any aggregation mechanism that integrates information from multiple acquisition positions into a common volumetric representation.

The three-dimensional volume refinement stage 412 includes any volumetric inference or refinement logic that transforms the aggregated representation into a useful output. This stage includes decoder-only networks, full three-dimensional U-Nets, monolithic encoder-decoder networks, three-dimensional transformers, diffusion-based models, variational autoencoders, GAN generators paired with discriminators, coordinate-based decoders such as NeRF-like decoders, and other volumetric learning approaches. The volumetric output 414 spans a spatial extent, in at least one dimension, exceeding the field of view of any single fluoroscopic frame.

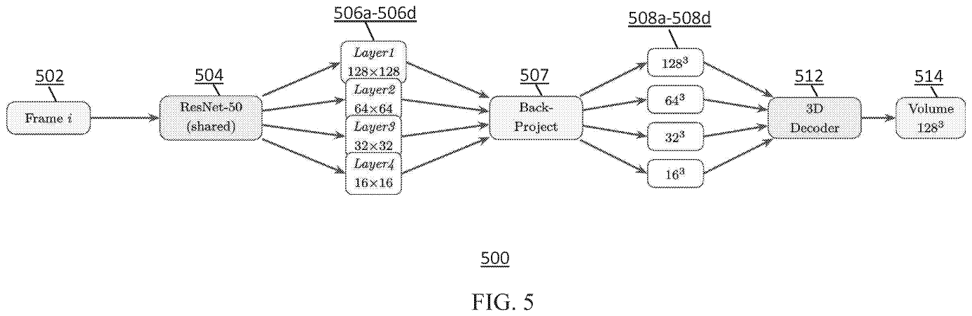

Referring to FIG. 5, a first reconstruction architecture 500 receives fluoroscopic frames 502 and processes the frames 502 through a shared two-dimensional encoder 504. The shared encoder 504 outputs multi-resolution feature maps 506a, 506b, 506c, 506d. Those feature maps are back-projected at step 507 into corresponding three-dimensional feature volumes 508a, 508b, 508c, 508d, which are combined by an aggregation block 510, and a decoder-only three-dimensional network 512 generates a volumetric output 514. The encoder 504 includes a ResNet-50 encoder as one representative implementation, but also includes other CNNs, transformers, hybrid architectures, and U-Net-like encoders. The multi-resolution feature maps 506a-506d correspond to selected spatial scales and semantic levels, and the architecture 500 provides a computational profile in which significant feature extraction occurs in two dimensions prior to volumetric decoding.

In one representative implementation of architecture 500, the shared two-dimensional encoder 504 is a ResNet-50 encoder that produces feature maps at multiple resolution levels. A selected feature-to-scale mapping routes one specific encoder level to each volumetric scale so that, for example, a first selected encoder layer contributes to a finest-resolution cube and progressively deeper encoder layers contribute to progressively coarser cubes. The decoder-only three-dimensional network 512 treats the resulting back-projected feature volumes 508a, 508b, 508c, 508d as skip-connected volumetric inputs and performs learned upsampling and refinement to generate the volumetric output 514. This implementation is computationally efficient because substantial feature extraction occurs in the two-dimensional domain before volumetric decoding.

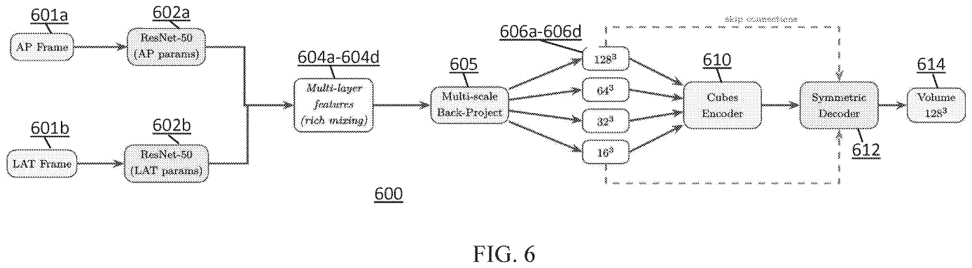

Referring to FIG. 6, a second reconstruction architecture 600 includes a first-view input 601a and a second-view input 601b, corresponding view-specific encoders 602a and 602b, mixed feature inputs 604a, 604b, 604c, 604d, back-projection step 605, cubes 606a, 606b, 606c, 606d, aggregation block 608, cubes encoder 610, cubes decoder 612, and volumetric output 614. The view-specific encoders 602a and 602b can share weights, partially share weights, or be independently parameterized so that orientation-specific image characteristics are preserved. The mixed feature inputs 604a-604d include selected channels from different encoder depths or views, and the cubes encoder 610 and cubes decoder 612 provide a higher-capacity volumetric stage that can make stronger use of multi-view translational acquisition data.

In one representative implementation of second reconstruction architecture 600, the view-specific encoders 602a and 602b are separate ResNet-50 encoders corresponding to AP and lateral image streams. The mixed feature inputs 604a, 604b, 604c, 604d are formed by drawing channels from multiple encoder depths at each three-dimensional scale so that each back-projected cube 606a, 606b, 606c, 606d can include both local-detail information and broader semantic context. The cubes encoder 610 processes the cubes from finest to coarsest resolution, and the corresponding cube is injected at each level transition, after which the cubes decoder 612 reconstructs or refines the volumetric output 614. This arrangement supports richer cross-scale and cross-view reasoning than a simpler decoder-only design.

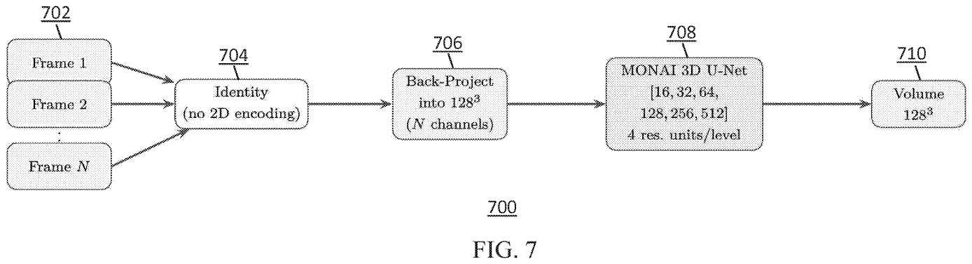

Referring to FIG. 7, a third reconstruction architecture 700 receives raw fluoroscopic image inputs 702 without a learned two-dimensional encoder. The raw inputs 702 are back-projected by a direct back-projection block 704 into an aggregated volumetric tensor 706, and a monolithic three-dimensional network 708 generates a volumetric output 710. The “raw” fluoroscopic image inputs 702 can still be calibrated, normalized, denoised, resized, or otherwise preprocessed while remaining image-like rather than feature-encoded. The monolithic three-dimensional network 708 includes a MONAI UNet, SegResNet, a volumetric transformer, or another encoder-decoder volumetric architecture that learns cross-frame anatomical consistency directly from the aggregated tensor 706.

In one representative implementation of third reconstruction architecture 700, the monolithic three-dimensional network 708 is a MONAI UNet or similar three-dimensional encoder-decoder having multiple channel stages, such as progressively increasing channel dimensions from finer to coarser volumetric resolutions, together with residual units or related volumetric refinement blocks at one or more stages. The raw fluoroscopic image inputs 702 are treated as image-like inputs rather than learned two-dimensional features, but those inputs still include calibrated, normalized, denoised, artifact-suppressed, or otherwise preprocessed fluoroscopic frames so long as they remain substantially raw-image representations for direct back-projection by block 704.

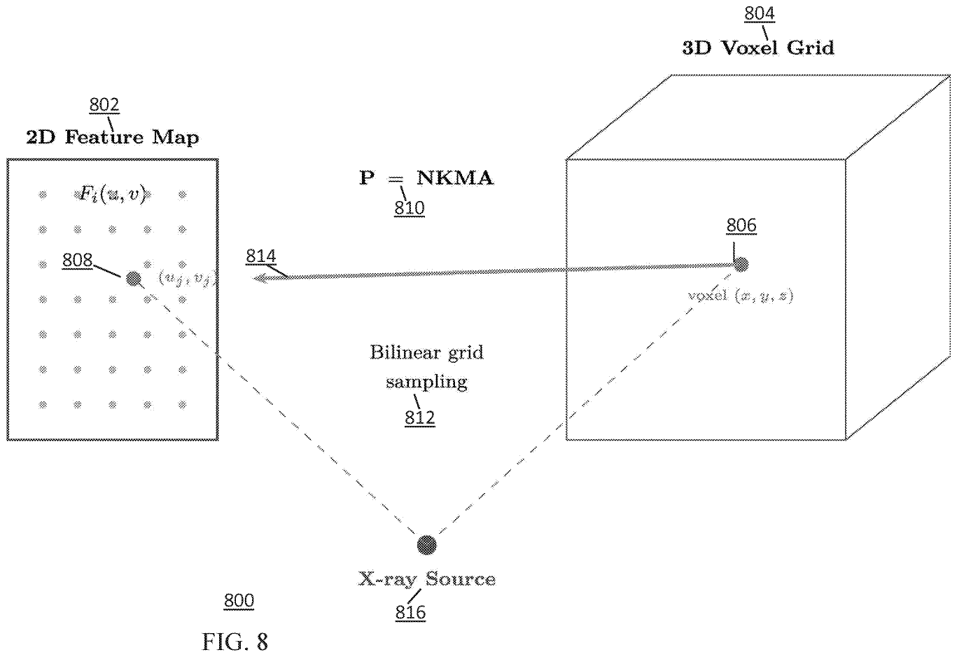

Referring to FIG. 8, a back-projection geometry arrangement 800 maps a two-dimensional feature map 802 into a three-dimensional voxel grid 804. A representative voxel sample point 806 in the voxel grid 804 is projected to a corresponding image coordinate 808 by a projection matrix 810, and a bilinear grid-sampling operation 812 samples the feature map 802 at the projected image coordinate 808 within an image plane 814, based on the imaging geometry defined relative to a camera or source coordinate frame 816. The feature map 802 includes a raw image plane, a learned feature tensor, a panoramic feature representation, or another two-dimensional sampling surface. The voxel grid 804 includes a regular Cartesian grid, a multi-scale grid, an anisotropic grid, an extended grid, a tiled grid, a sliding-window grid, or another three-dimensional sampling structure. The image coordinate 808 includes a detector-space coordinate, normalized coordinate, pixel coordinate, or projected feature-space coordinate. The projection matrix 810 includes a composite or partial transform that relates volumetric coordinates to image-plane coordinates. In one representative form, the matrix is expressed as P=NKiMiA, where A maps voxel-grid coordinates to world coordinates, Mi is the extrinsic pose matrix for frame i, Ki is the intrinsic camera matrix for frame i, and N normalizes detector or image coordinates into the coordinate range expected by the grid-sampling operation. Equivalent transform formulations remain within the present disclosure.

The back-projection geometry arrangement 800 includes deterministic back-projection, differentiable back-projection, sparse voxel lifting, dense volumetric lifting, ray-based lifting, or another geometry-aware image-to-volume mapping. The bilinear grid-sampling operation 812 includes bilinear interpolation, nearest-neighbor interpolation, higher-order interpolation, differentiable interpolation, or equivalent sampling logic. Voxels projecting outside the valid extent of the image plane 814 can be zero-padded, masked, clipped, ignored, or otherwise handled. The differentiable form allows gradients to propagate through the back-projection operation during training.

In one representative formulation, the projection matrix 810 is expressed as P=NKiMiA, interpreted right to left. The transform A maps a voxel-grid location in the three-dimensional voxel grid 804 into world-space coordinates, Mi maps the world-space coordinates into the camera or source coordinate frame 816 for frame i, Ki maps the frame-specific camera-space coordinates into detector or image-plane coordinates on image plane 814, and N normalizes the resulting coordinates into the range expected by the grid-sampling operation 812. Thus, each representative voxel sample point 806 is projected to a corresponding image coordinate 808, and the value of the two-dimensional feature map 802 is sampled at that coordinate to populate the three-dimensional volumetric representation. Equivalent transform chains remain within the present disclosure.

The geometry-aware back-projection arrangement 800 is not merely an abstract mathematical transformation, but instead uses machine-specific imaging geometry to map volumetric sample locations to image-domain coordinates based on the physical configuration of the fluoroscopic image acquisition system for each frame. In representative implementations, the projection matrix 810 encodes intrinsic detector geometry, extrinsic frame pose, and voxel-grid-to-world alignment so that the value sampled from the two-dimensional feature map 802 depends on the actual source-detector arrangement and patient-relative imaging geometry present during fluoroscopic acquisition. Geometry-aware back-projection as used herein therefore includes generalized back-projection based on arbitrary calibrated imaging poses, intrinsic transforms, and voxel-grid definitions, including the generalized geometry-adapted back-projection approaches described in incorporated U.S. Pat. No. 12,444,127 for arbitrary X-ray imaging-system poses.

In representative implementations, the voxel grid 804 is defined in a patient-referenced or reconstruction-referenced coordinate frame so that projected voxel coordinates fall within the usable field of view of the image plane 814 for the contributing fluoroscopic views. The incorporated reconstruction family materials describe representative use of a registration transform to define voxel-grid coordinates, align basis vectors, and project voxel coordinates onto X-ray images using calibration matrices, and those disclosures provide additional support for the geometry-adapted voxel-grid and back-projection operations described herein.

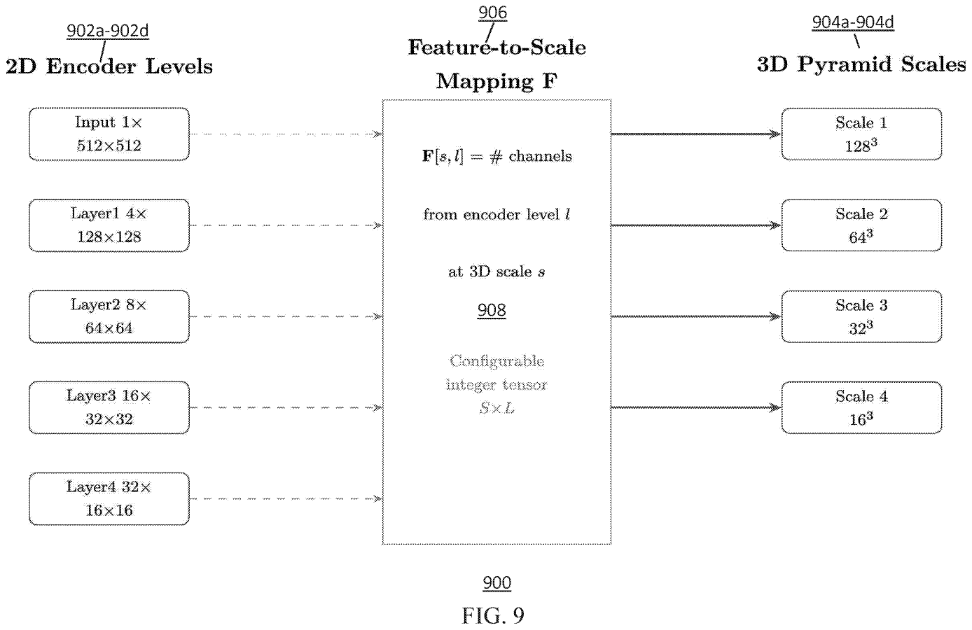

Referring to FIG. 9, a multi-scale back-projection pyramid 900 includes two-dimensional feature levels 902a, 902b, 902c, 902d, corresponding three-dimensional scale volumes 904a, 904b, 904c, 904d, a feature-to-scale mapping 906, and routing connections 908. The feature-to-scale mapping 906 includes a fixed routing design, configurable routing design, or learned routing design that determines how channels from the two-dimensional levels are allocated into one or more volumetric scales. In one representative implementation, a mapping tensor specifies how many channels from each two-dimensional encoder level are sent to each three-dimensional scale. The routing connections 908 therefore include one-to-one, one-to-many, many-to-one, dense, sparse, gated, or weighted paths. This arrangement provides a flexible bridge between two-dimensional multi-resolution encoders and three-dimensional hierarchical volumetric networks.

In one representative implementation, the feature-to-scale mapping 906 is represented by a configurable mapping tensor whose entries specify how many feature channels from a given two-dimensional feature level 902a, 902b, 902c, 902d are back-projected into a given three-dimensional scale volume 904a, 904b, 904c, 904d. A zero-valued entry indicates that a particular two-dimensional level is not used for a particular volumetric scale, while a non-zero entry indicates that one or more channels are routed through the corresponding routing connection 908. This arrangement allows computation and memory usage to be adjusted without changing the underlying geometry-aware reconstruction concept.

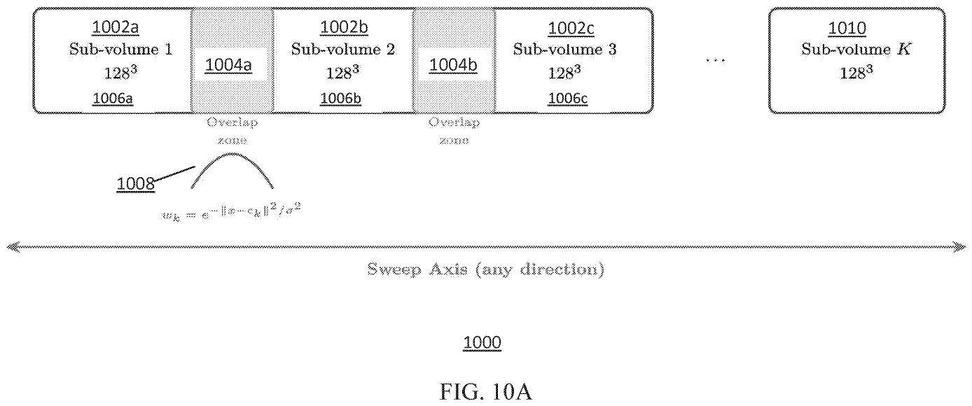

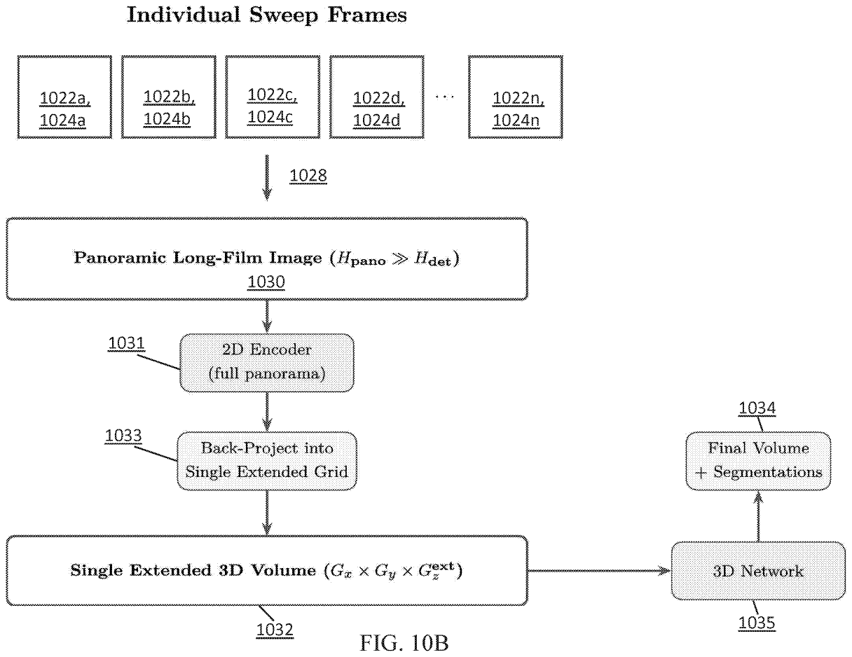

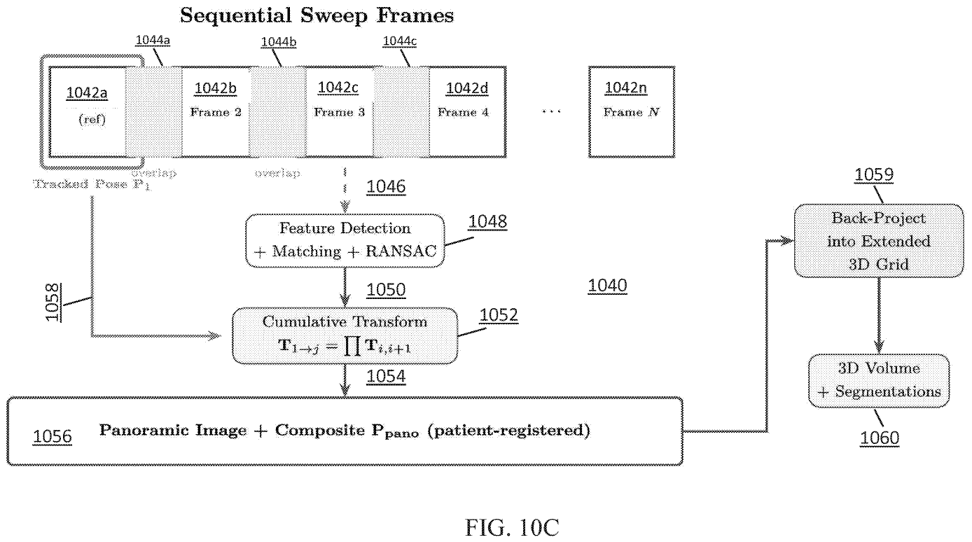

The present disclosure includes several extended-field-of-view reconstruction approaches. Referring to FIG. 10A, a tiled reconstruction arrangement 1000 includes reconstructed sub-volumes 1002a, 1002b, 1002c, overlap zones 1004a, 1004b, sub-volume centers 1006a, 1006b, 1006c, a Gaussian blending profile 1008, and a final extended reconstructed volume 1010. In this approach, the sweep is partitioned into overlapping sub-regions, each sub-region is reconstructed separately, and neighboring sub-volumes are blended in the overlap zones 1004a, 1004b. Each sub-volume 1002a, 1002b, 1002c can be produced from a corresponding subset of AP, lateral, or other sweep frames and then placed in patient space prior to blending. The Gaussian blending profile 1008 is one example of a weighted averaging profile, and the present disclosure also includes other tapering, confidence-weighted, or learned blending schemes. The final extended reconstructed volume 1010 includes a merged, blended, stitched, or otherwise unified volume spanning an anatomical extent larger than any one sub-volume.

In one representative implementation, each reconstructed sub-volume 1002a, 1002b, 1002c has a local volumetric size such as approximately 128\hat{ }3 (i.e., 128³) voxels at about 1 mm spacing or approximately 256\hat{ }3 voxels at about 0.5 mm spacing, although larger or smaller volumes remain within the present disclosure. In the overlap zones 1004a, 1004b, voxel contributions from neighboring sub-volumes are combined according to spatial weighting centered on the corresponding sub-volume centers 1006a, 1006b, 1006c. A representative weighting profile uses a Gaussian blending profile 1008 so that each voxel value in the final extended reconstructed volume 1010 is formed as a weighted combination of the overlapping sub-volume values, with weights that decrease with distance from the center of each contributing sub-volume. This blending reduces seam artifacts and improves continuity across the extended volume.

In another approach, all frames are back-projected into a single extended volumetric grid whose extent along the sweep direction corresponds to the total sweep length or another extended anatomical dimension. Such an extended grid can be processed in one pass, in overlapping windows, or through recurrent extension depending on memory constraints. The present disclosure also includes recurrent incremental extension in which groups of frames are processed sequentially while a latent state tensor carries context forward so that the volume extends progressively without requiring the full anatomy to reside in memory simultaneously.

Referring to FIG. 10B, a tracker-based panoramic stitching mode 1020 includes sequential fluoroscopic frames 1022a, 1022b, 1022c, 1022d, 1022n, tracked frame offsets 1024a, 1024b, 1024c, 1024d, 1024n, a panoramic image canvas 1026, blended overlap regions 1028, a panoramic image 1030, a two-dimensional encoder 1031, back-projection step 1033 into a single extended voxel grid 1032, a three-dimensional network 1035, and a volumetric reconstruction output 1034. In this mode, the frame offsets are provided by the tracking system so that the panoramic image 1030 is assembled using known geometry. Separate panoramas can be formed for AP and lateral sweeps and then encoded, back-projected, and processed jointly or separately. The panoramic image 1030 can provide global two-dimensional context across a long anatomical extent before volumetric inference, which improves continuity and reduces seam artifacts.

In a representative implementation of tracker-based panoramic stitching mode 1020, each frame 1022a, 1022b, 1022c, 1022d, 1022n is placed in the panoramic image canvas 1026 according to its corresponding tracked frame offset 1024a, 1024b, 1024c, 1024d, 1024n, such that the sweep-direction extent of the resulting panoramic image 1030 corresponds to the total swept extent projected onto the detector plane. A similar panoramic image can be generated for each sweep orientation, including AP, lateral, or oblique sweeps. The panoramic image 1030 is then processed directly or through the two-dimensional encoder 1031, back-projected at step 1033 into the extended voxel grid 1032, and refined by the three-dimensional network 1035 to produce the volumetric reconstruction output 1034. This approach allows the system to exploit global two-dimensional anatomical context over the full sweep extent before volumetric inference.

Referring to FIG. 10C, an image-based panoramic stitching mode 1040 includes sequential fluoroscopic frames 1042a, 1042b, 1042c, 1042d, 1042n, overlap regions 1044a, 1044b, 1044c, detected image features 1046, matched correspondences 1048, pairwise transforms 1050, a cumulative transform chain 1052, a panoramic image canvas 1054, a panoramic image 1056, an optional single tracked reference pose 1058, back-projection step 1059, and a reconstructed extended volume 1060. The fluoroscopic-specific stitching challenges addressed by this mode include low contrast-to-noise ratio, repetitive vertebral anatomy, divergent cone-beam projection geometry, and the utility of engineered calibration features visible in every frame. The image-based panoramic mode therefore broadens the workflow to include reconstruction from limited tracked geometry while still enabling panoramic compositing and extended-field volumetric inference.

Fluoroscopic panoramic stitching in mode 1040 differs materially from ordinary optical panorama stitching because the fluoroscopic frames 1042a, 1042b, 1042c, 1042d, 1042n often exhibit low contrast-to-noise ratio, repetitive vertebral anatomy, and cone-beam projection distortion that can make ordinary photographic stitching techniques unreliable. Repetitive structures such as vertebral bodies, pedicles, and spinous processes can create ambiguity in the matched correspondences 1048, and spatially varying magnification can limit the accuracy of simple translation-only models. For this reason, the detected image features 1046 can include both natural anatomical features and engineered radiopaque calibration-device features that remain visible in every frame, thereby improving robustness of the pairwise transforms 1050 and the cumulative transform chain 1052. The optional single tracked reference pose 1058 further constrains drift by anchoring the image-derived transform chain to patient space.

In some implementations, the inter-frame geometric transformation is selected to accommodate the depth-dependent magnification inherent in divergent cone-beam projection geometry. The transform model may be anchored to a representative anatomical depth determined from the calibration target geometry, the tracked fiducial geometry, or an average source-to-object distance estimated from the imaged anatomy. Alternatively, the transform may be parameterized as a learned warp predicted by a trained neural network that absorbs cone-beam parallax and other projection-geometry-dependent distortion into a single image-to-image mapping. In either approach, the resulting inter-frame transform produces a panoramic composite in which spatially varying magnification across the detector field is compensated to within a tolerance acceptable for downstream geometry-aware back-projection.

In some implementations, a “single extended three-dimensional voxel grid” relates to a grid whose extent along the sweep axis or another anatomical dimension is larger than the grid used for a local or vertebra-sized reconstruction and that spans the overall sweep length or another extended anatomical dimension. In some implementations, a “plurality of tiled sub-volumes” includes the partitioned-overlap approach in which local volumetric reconstructions are blended into a larger final volume. In some implementations, a “panoramic image” includes a long-film composite image assembled from sequential fluoroscopic frames using tracked offsets, image-derived transforms, or both, and includes AP panoramas, lateral panoramas, and panoramas from additional orientations.

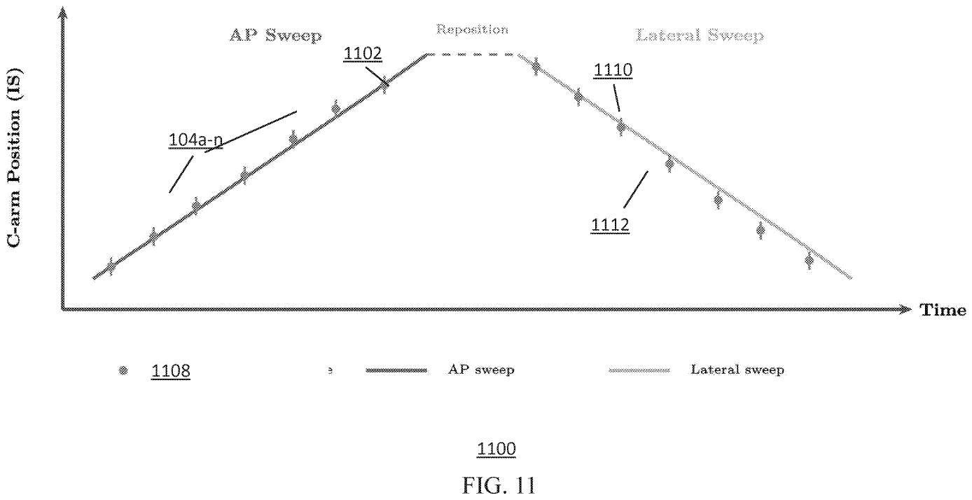

Referring to FIG. 11, an acquisition timing diagram 1100 includes a translational position trace 1102 plotted as a function of time, synchronized X-ray pulse and pose capture events 1108a, 1108b, 1108c, 1108n, a synchronization relationship 1110, and sweep intervals 1112a, 1112b. In one representative implementation, sweep interval 1112a corresponds to an AP sweep and sweep interval 1112b corresponds to a lateral sweep. The event series 1108a-1108n represents combined image-acquisition and associated pose-capture events, whether achieved by shared hardware triggers, timestamp alignment, interpolation from neighboring measurements, or another synchronization mechanism. This broad treatment covers a wide range of acquisition control schemes while preserving temporal coherence between image data and spatial metadata.

In representative extended-field workflows, the total number of synchronized events 1108a, 1108b, 1108c, 1108n corresponds to an acquisition sequence of more than four frames and often to approximately 10 to 100 or more fluoroscopic frames depending on anatomy size, desired overlap, and frame rate. This acquisition regime differs materially from classical CBCT workflows, which often rely on dozens to hundreds of projections acquired at one location during an orbital sweep, and also differs from sparse-view neural-reconstruction approaches that use only a small number of static projections. The acquisition timing diagram 1100 therefore represents a translational acquisition regime that is specifically adapted for extended-field fluoroscopic volumetric reconstruction.

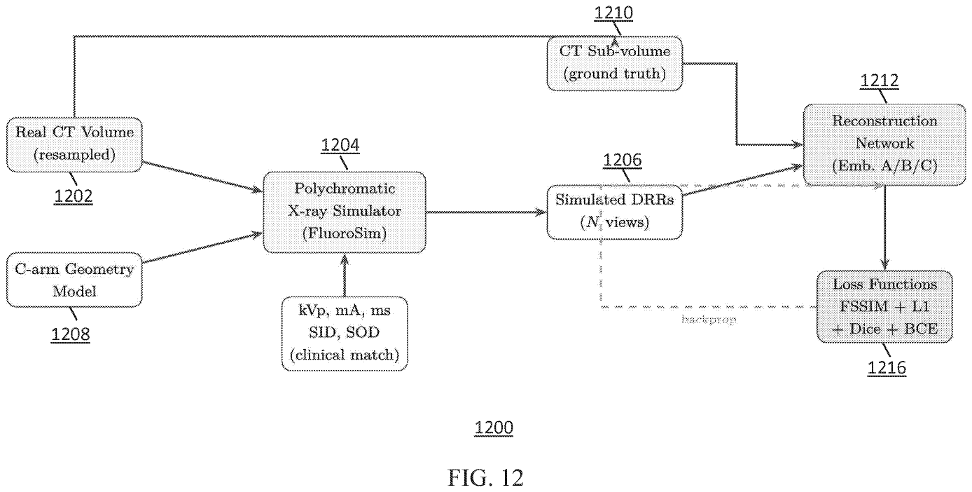

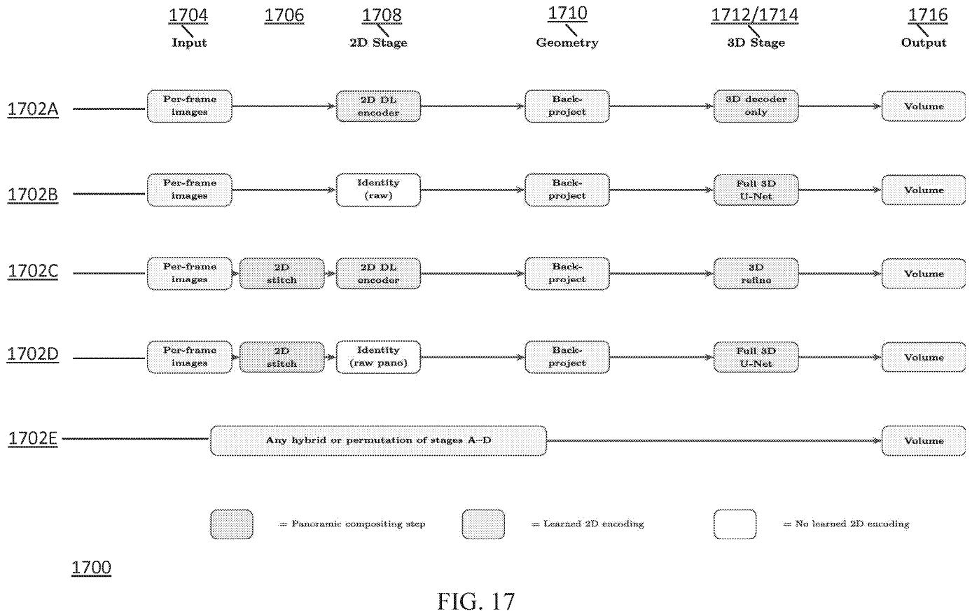

Referring to FIG. 12, a training pipeline 1200 includes a CT volume dataset 1202, a physics-based DRR simulator 1204, simulated fluoroscopic views 1206, a projection-geometry generator 1208, a training sample generator 1210, a neural network training engine 1212, a trained reconstruction model 1214, and a loss computation block 1216. The CT volume dataset 1202 includes real CT volumes resampled to a target isotropic spacing and optionally curated or augmented for anatomy of interest. The projection-geometry generator 1208 includes a C-arm geometry model capable of reproducing translational and non-isocentric acquisition geometries. The physics-based DRR simulator 1204 includes a polychromatic X-ray simulator configured according to clinically matched parameters such as tube voltage, tube current, pulse width, source-to-image distance, and source-to-object distance. The simulated fluoroscopic views 1206 include AP, lateral, oblique, translationally spaced, or otherwise generated views corresponding to the target acquisition geometries. The training sample generator 1210 includes landmark-centered or anatomy-centered cropping, CT sub-volume extraction, paired-input generation, augmentation, and balancing. The neural network training engine 1212 trains any of the disclosed reconstruction model families, including the architectures of FIGS. 5-7 and the variants of FIG. 17. The loss computation block 1216 includes one or more reconstruction losses, segmentation losses, perceptual losses, structural similarity losses, adversarial losses, geometric-consistency losses, or combinations thereof. The trained reconstruction model 1214 therefore includes models corresponding to multiple reconstruction variants and output types, including CT-like reconstruction, tissue segmentation, metal segmentation, anatomical-part segmentation, or combinations thereof.

In one representative training workflow, the CT volume dataset 1202 includes real clinical CT volumes resampled to a target isotropic voxel spacing, such as about 1.0 mm or about 0.5 mm. The projection-geometry generator 1208 includes a C-arm geometry model that defines intrinsic and extrinsic parameters for each simulated fluoroscopic view, including AP, lateral, oblique, non-isocentric, or otherwise perturbed translational acquisition geometries. The physics-based DRR simulator 1204 includes a polychromatic X-ray simulator that models attenuation using NIST cross-section data together with beam hardening, detector response, and, in some implementations, scatter estimation. The simulated fluoroscopic views 1206 are formed using clinically matched acquisition parameters, such as tube voltage, tube current, pulse width, source-to-image distance, and source-to-object distance. The training sample generator 1210 can select a vertebral level or other anatomical landmark, crop a corresponding CT sub-volume, and generate paired DRR inputs and CT targets for the neural network training engine 1212. In some implementations, the center of the CT sub-volume is adjusted to a least-squares closest point relative to the central projection rays of the participating views so that the volumetric target remains consistent with the simulated image geometry. The loss computation block 1216 can include a weighted combination of Fourier-domain structural similarity, L1 loss, perceptual loss, Dice loss, binary cross-entropy, adversarial loss, or other reconstruction and segmentation objectives. The trained reconstruction model 1214 therefore includes a model capable of mapping geometrically consistent fluoroscopic inputs to volumetric outputs under translational sweep conditions.

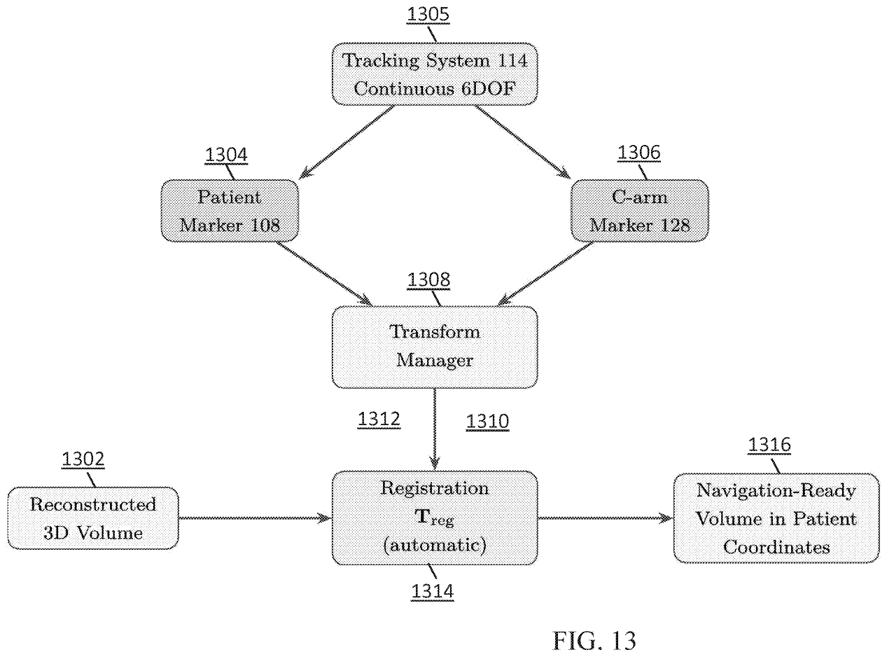

Referring to FIG. 13, an automatic registration pipeline 1300 includes a reconstructed volume 1302, a patient marker pose input 1304, a tracking system 1305, an imaging-system marker pose input 1306, a transform manager 1308, a patient coordinate system 1310, a volume coordinate system 1312, a registration transform 1314, and a navigation-ready output 1316. The transform manager 1308 computes or refines the spatial relationship between the patient coordinate system 1310 and the volume coordinate system 1312 using tracked or derived marker geometry, beam geometry, calibration-aware transforms, or chained transforms derived from acquisition and reconstruction. The resulting registration transform 1314 is a machine-level spatial transform that places the reconstructed volume 1302 into the same operative coordinate frame used by tracked instruments and navigation logic, rather than merely providing a display preference or abstract anatomical annotation. The navigation-ready output 1316 therefore includes a registered volume, registered labels, display overlays, tool guidance outputs, or another patient-referenced output that is directly usable by a surgical navigation workflow. The volumetric output is automatically registered to a patient coordinate system via spatial tracking measurements of the first and second fiducial markers, the registration being independent of any preoperative volumetric imaging study of the patient and any manual intraoperative landmark-correspondence step.

In representative implementations, the transform manager 1308 computes the registration transform 1314 using tracked source positions, detector-related geometry, central-beam directions, calibration-aware transforms, or combinations thereof to define the relationship between the patient coordinate system 1310 and the volume coordinate system 1312. The tracking system 1305 supplies the patient marker pose input 1304 and imaging-system marker pose input 1306, and the transform manager 1308 can derive an orthogonal basis or equivalent patient-referenced volumetric frame from those inputs together with the acquisition geometry. This environment is further supported by incorporated U.S. Pat. No. 12,361,631, entitled “System and Method for Generation of Registration Transform for Surgical Navigation,” issued Jul. 15, 2025, which describes representative techniques for constructing patient-referenced coordinate systems and registration transforms for surgical navigation, and by related biplanar-reconstruction family materials, which describe volumetric placement, source-detector geometry, and navigation-ready transformation logic applicable to the reconstructed volume 1302 and the navigation-ready output 1316.

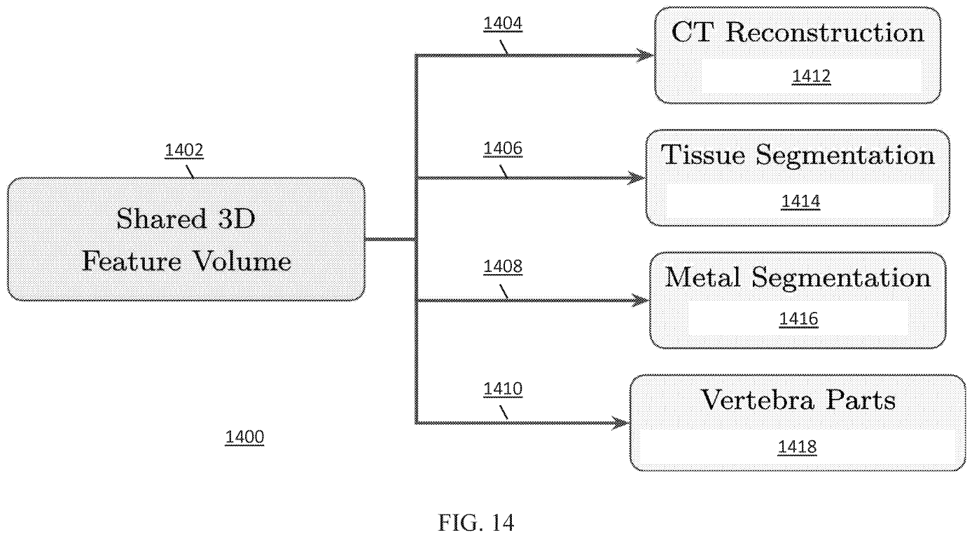

Referring to FIG. 14, a multi-head output architecture 1400 includes a final three-dimensional feature volume 1402, a CT-like reconstruction head 1404, a tissue segmentation head 1406, a metal segmentation head 1408, an anatomical-part segmentation head 1410, a CT-like volume output 1412, a tissue label output 1414, a metal mask output 1416, and an anatomical-part label output 1418. The CT-like reconstruction head 1404 generates image-intensity-like volumetric data, represented as the CT-like volume output 1412. The tissue segmentation head 1406 generates semantic classes such as bone, soft tissue, air, disc, canal, or other anatomy-related labels, represented as the tissue label output 1414. The metal segmentation head 1408 identifies metallic implants or high-density foreign material, represented as the metal mask output 1416. The anatomical-part segmentation head 1410 identifies vertebral bodies, processes, canals, discs, or other substructures, represented as the anatomical-part label output 1418. More or fewer heads can be used, and additional heads for confidence, artifact localization, alignment quality, or other procedural needs remain within the present disclosure.

In one representative implementation, the CT-like reconstruction head 1404 generates a volumetric intensity output resembling intraoperative CBCT or O-arm image quality, the tissue segmentation head 1406 predicts classes including bone, soft tissue, air, disc, and spinal canal, the metal segmentation head 1408 identifies metallic implants such as screws, rods, cages, or other hardware, and the anatomical-part segmentation head 1410 predicts vertebral substructures including vertebral body, canal, disc, and posterior elements or processes. These concrete examples are representative only, and the output heads remain configurable for different anatomical targets, clinical tasks, and downstream navigation or analysis uses.

Referring to FIG. 17, a pipeline variant taxonomy 1700 includes pipeline variants 1702A, 1702B, 1702C, 1702D, 1702E, a per-frame path 1704, a panoramic-first path 1706, a learned two-dimensional encoding option 1708, a raw-image option 1710, a decoder-only three-dimensional path 1712, an encoder-decoder three-dimensional path 1714, and one or more volume outputs 1716. These pipeline variants are representative examples rather than an exhaustive list. The per-frame path 1704 includes workflows in which individual frames are processed before volumetric integration. The panoramic-first path 1706 includes workflows in which frames are first composited into one or more panoramic images before volumetric lifting. The learned two-dimensional encoding option 1708 includes any feature-learning stage applied before volumetric inference, while the raw-image option 1710 includes workflows operating substantially directly on image-like inputs. The one or more volume outputs 1716 include reconstructed volumes, segmentation-bearing volumes, registered volumes, or otherwise usable volumetric products generated by different pipeline pathways.

In one representative taxonomy, pipeline variant 1702A corresponds to per-frame two-dimensional encoding followed by back-projection, aggregation, and three-dimensional refinement; pipeline variant 1702B corresponds to back-projection of raw or unencoded image data followed by full three-dimensional encoding and decoding; pipeline variant 1702C corresponds to panoramic compositing followed by two-dimensional encoding, back-projection, and three-dimensional refinement; pipeline variant 1702D corresponds to panoramic compositing followed by back-projection of raw panoramic image data and full three-dimensional processing; and pipeline variant 1702E corresponds to hybrids, permutations, or combinations of the foregoing. The one or more volume outputs 1716 therefore include results produced by multiple valid orderings of the same core geometry-aware reconstruction principles.

The systems and methods described above are implemented in software, hardware, firmware, or combinations thereof. The disclosed neural-network components are implemented using convolutional neural networks, transformers, hybrid models, diffusion models, autoencoders, adversarial networks, coordinate-based networks, or other machine-learning architectures. The tracking, compositing, and calibration operations are implemented using deterministic algorithms, learned algorithms, or hybrid algorithms. Features described in connection with one figure or one processing mode are combinable with features described in connection with other figures or other processing modes unless technically incompatible. For example, a tiled volumetric approach can be combined with sparse tracking, a panoramic approach can be combined with inline calibration, and a monolithic three-dimensional network can be combined with image-based inter-frame registration. This specification is therefore intended to support claims directed to systems, methods, non-transitory computer-readable media, and subcombinations thereof across the full extended-field fluoroscopic volumetric reconstruction ecosystem described and illustrated by FIGS. 1A-19.

The present disclosure therefore provides an improved fluoroscopic imaging system. The present system enables standard fluoroscopic equipment to generate extended-field three-dimensional volumetric outputs from one or more continuous translational sweeps, by deriving frame-specific projection geometry from tracked or image-derived positional data, by reconstructing anatomically coherent volumes through geometry-aware back-projection and trained three-dimensional neural refinement, and by automatically registering the resulting volume to patient space without requiring a preoperative CT scan or manual intraoperative registration. The resulting workflow transforms machine-acquired fluoroscopic image sequences and associated spatial metadata into a new patient-referenced volumetric representation that supports multiplanar viewing, segmentation, navigation, guidance, and related intraoperative uses.

This application is also related to the following commonly owned patents and patent applications, each of which is incorporated herein by reference in its entirety:
U.S. Pat. No. 12,361,631, entitled “System and Method for Generation of Registration Transform for Surgical Navigation,” issued Jul. 15, 2025;
U.S. Pat. No. 12,444,127, entitled “System and Method for Reconstruction of 3D Volumes from Biplanar Radiographic Images,” issued Oct. 14, 2025;
U.S. Pat. No. 12,462,469, entitled “Wire-Based Calibration Apparatus for X-ray Imaging Systems,” issued Nov. 4, 2025;
U.S. patent application Ser. No. 18/974,434, entitled “Omni-View Unique Tracking Marker,” filed Dec. 9, 2024, published as U.S. Patent Application Publication 2025/0186163A1.

What is claimed is:

1. A system for reconstructing a three-dimensional volumetric image of an extended anatomical region of a patient exceeding the native field of view of a fluoroscopic detector, comprising:
(a) a fluoroscopic image acquisition system configured to capture a sequence of N fluoroscopic frames during one or more continuous translational sweeps along an extended anatomical region of interest, wherein N>4, and wherein successive frames have at least partial spatial overlap;
(b) a first fiducial marker rigidly mounted to the fluoroscopic image acquisition system;
(c) a second fiducial marker affixable to the patient proximate the anatomical region of interest;
(d) a spatial tracking system comprising at least one spatial sensing device and configured to track both the first and second fiducial markers during at least a portion of the sequence of fluoroscopic frames, thereby generating six-degree-of-freedom positional data for at least one frame in the sequence, the positional data being usable, in combination with intrinsic camera parameters of the fluoroscopic image acquisition system, to derive a projection matrix, wherein a projection matrix for each of the remaining frames is derived from the positional data by at least one of: direct tracking measurement, interpolation between tracked frames, image-based inter-frame registration, or any combination thereof;
(e) a processor configured to execute a trained neural network model that:
(i) produces, from the N fluoroscopic frames, one or more two-dimensional representations, each two-dimensional representation being selected from: a raw fluoroscopic image, a composited panoramic image formed by spatially registering and blending a plurality of sequential frames, or learned feature maps extracted by a trained two-dimensional deep learning network operating on individual fluoroscopic frames or on a composited panoramic image;
(ii) back-projects the one or more two-dimensional representations into one or more three-dimensional voxel grids using projection matrices derived from the positional data; and
(iii) produces a volumetric output by processing the back-projected representations through a trained three-dimensional deep learning network; and
(f) wherein the volumetric output spans a spatial extent, in at least one dimension, exceeding the field of view of any single fluoroscopic frame, and is automatically registered to a patient coordinate system via spatial tracking measurements of the first and second fiducial markers, the registration being independent of any preoperative volumetric imaging study of the patient and any manual intraoperative landmark-correspondence step.

2. The system of claim 1, wherein the two-dimensional representation comprises multi-resolution feature maps extracted by a deep learning encoder network producing features at a plurality of spatial resolution levels with respective downsampling factors.

3. The system of claim 2, wherein the deep learning encoder network is selected from the group comprising a ResNet-family convolutional neural network, a VGG-family network, an EfficientNet, a DenseNet, a MobileNet, a ConvNeXt, a ViT, a DeiT, a Swin Transformer, a PVT, a CoAtNet, a MaxViT, and a hybrid CNN-transformer architecture, producing feature maps at a plurality of resolution levels.

4. The system of claim 1, wherein the two-dimensional representation comprises the raw fluoroscopic image without learned two-dimensional encoding.

5. The system of claim 1, wherein the back-projection operates at multiple spatial scales, producing a plurality of three-dimensional cubes at progressively coarser resolutions, wherein different two-dimensional feature levels are selectively routed to different three-dimensional scales via a configurable feature-to-scale mapping.

6. The system of claim 1, wherein the three-dimensional deep learning network comprises a U-Net with both a three-dimensional encoder and a three-dimensional decoder, wherein the encoder processes multi-scale back-projected cubes from finest to coarsest resolution with skip connections, and the decoder upsamples with symmetric skip connections.

7. The system of claim 1, wherein the back-projection produces a single three-dimensional voxel grid at a single spatial scale, and the three-dimensional deep learning network comprises a monolithic three-dimensional encoder-decoder network selected from the group comprising a 3D convolutional neural network, a MONAI UNet, a Vision Transformer, a UNETR, a SwinUNETR, a SegResNet, and a hybrid CNN-transformer architecture, the monolithic network receiving the single back-projected voxel grid and performing both encoding and decoding to produce the volumetric output.

8. The system of claim 1, wherein the volumetric output simultaneously comprises a CT-like reconstruction volume, a multi-class tissue segmentation volume, a binary metal segmentation volume, and an anatomical part segmentation volume, each produced by a separate output head from a shared three-dimensional feature volume.

9. The system of claim 1, wherein each fluoroscopic frame contains a radiopaque calibration device comprising a plurality of radiopaque markers at known three-dimensional coordinates, and wherein per-frame intrinsic and extrinsic camera parameters are computed from detected marker features by establishing two-dimensional-to-three-dimensional point correspondences and solving the perspective-n-point problem.

10. The system of claim 9, wherein radiopaque calibration device artifacts are digitally removed from each frame prior to computation of the two-dimensional representation.

11. The system of claim 9, wherein the radiopaque calibration device comprises a radiopaque geometry selected from the group consisting of a wire-based device having crossover features or gap features, a spherical-marker device having centroid features, a coded-marker arrangement, a grid pattern, a plate pattern, and combinations thereof, the radiopaque geometry providing known three-dimensional feature locations visible in the fluoroscopic frame for per-frame inline calibration.

12. The system of claim 1, wherein the processor is further configured to, prior to back-projection, composite the sequential fluoroscopic frames from each sweep orientation into a panoramic image by spatially registering each frame using its per-frame projection matrix and blending overlapping regions using spatially weighted averaging, and to back-project the panoramic image into a single extended three-dimensional voxel grid spanning the full swept anatomy without sub-volume tiling.

13. The system of claim 1, wherein the one or more continuous translational sweeps comprise at least one sweep along an axis that is not parallel to the patient's longitudinal axis, and the volumetric output spans a spatial extent along the sweep direction exceeding the field of view of any single fluoroscopic frame.

14. The system of claim 1, wherein the three-dimensional deep learning network comprises a generative model selected from the group consisting of a generative adversarial network, a diffusion model, a variational autoencoder, and a neural radiance field decoder.

15. The system of claim 1, wherein the spatial tracking system generates six-degree-of-freedom positional data for every frame in the sequence, each frame having a temporally synchronized pose measurement.

16. A system for generating a three-dimensional volumetric image of an extended anatomical region exceeding the native field of view of a fluoroscopic detector, comprising:
a fluoroscopic image acquisition system configured to capture a sequence of N fluoroscopic frames during one or more continuous translational sweeps along an extended anatomical region of interest, wherein N>4 and successive frames have at least partial spatial overlap; and
a processor configured to:
composite sequential fluoroscopic frames from at least one sweep orientation into a panoramic image by spatially registering and blending a plurality of sequential frames;
back-project the panoramic image into a single extended three-dimensional voxel grid spanning the full swept anatomy; and
produce a volumetric output by processing the single extended three-dimensional voxel grid through a trained three-dimensional deep learning network,
wherein the volumetric output spans a spatial extent exceeding the field of view of any single fluoroscopic frame.

17. The system of claim 16, wherein the panoramic image is processed by a two-dimensional encoder network to produce panoramic feature maps prior to back-projection, thereby enabling the encoder network to learn long-range anatomical relationships spanning the full extent of the swept anatomy.

18. The system of claim 16, wherein the spatial registration of sequential fluoroscopic frames for compositing the panoramic image is determined from image content by detecting and matching features in overlapping regions between adjacent frames and estimating inter-frame geometric transformations, rather than from per-frame spatial tracking measurements.

19. The system of claim 18, wherein a six-degree-of-freedom pose is captured by a spatial tracking system for only a single reference frame in the sweep sequence, and projection matrices for all remaining frames are derived by composing the image-derived inter-frame geometric transformations into a cumulative transform chain from the single reference pose.

20. The system of claim 16, wherein each fluoroscopic frame is acquired as a digital image in a format selected from the group consisting of a DICOM image, a cine digital sequence, a raw detector output, and any other digital image format, the digital image preserving at least 10 bits of the bit depth of the fluoroscopic detector.

21. A method for generating a three-dimensional volumetric image of an extended anatomical region that exceeds the native field of view of a fluoroscopic detector, comprising:
(a) acquiring a sequence of N fluoroscopic frames, wherein N is greater than 4, during one or more continuous translational sweeps along an extended anatomical region of interest, wherein successive frames have at least partial spatial overlap, the fluoroscopic frames acquired in any image format including native digital formats or digitized analog video;
(b) tracking, via a spatial tracking system comprising at least one spatial sensing device, a first fiducial marker mounted to the fluoroscopic image acquisition system and a second fiducial marker affixed to a patient, thereby generating six-degree-of-freedom positional data for at least one frame in the sequence, the positional data being usable, in combination with intrinsic camera parameters of the fluoroscopic image acquisition system, to derive a projection matrix for each of the N frames by at least one of: direct tracking measurement, interpolation between tracked frames, image-based inter-frame registration, or any combination thereof;
(c) producing, from the N fluoroscopic frames, one or more two-dimensional representations, each two-dimensional representation being selected from: a raw fluoroscopic image, a composited panoramic image formed by spatially registering and blending a plurality of sequential frames, or learned feature maps extracted by a trained two-dimensional deep learning network operating on individual fluoroscopic frames or on a composited panoramic image;
(d) back-projecting the one or more two-dimensional representations into one or more three-dimensional voxel grids using projection matrices derived from the positional data;
(e) producing a volumetric output by processing the back-projected representations through a trained three-dimensional deep learning network, the volumetric output spanning a spatial extent, in at least one dimension, exceeding the field of view of any single fluoroscopic frame; and
(f) automatically registering the volumetric output to a patient coordinate system via spatial tracking measurements of the first and second fiducial markers, the registration being independent of any preoperative volumetric imaging study of the patient and any manual intraoperative landmark-correspondence step.

22. The method of claim 21, wherein the volumetric output is reconstructed as a plurality of tiled sub-volumes blended in overlap regions using Gaussian-weighted averaging.

23. The method of claim 21, further comprising, prior to back-projection, compositing the sequential fluoroscopic frames from each sweep orientation into a panoramic image by spatially registering each frame and blending overlapping regions, and back-projecting the panoramic image into a single extended three-dimensional voxel grid spanning the full swept anatomy.

24. The method of claim 23, wherein the spatial registration of sequential frames for compositing the panoramic image is determined from image content by detecting and matching features in overlapping regions between adjacent frames rather than from per-frame spatial tracking measurements, and wherein a six-degree-of-freedom pose is captured for only a single reference frame in the sweep sequence.

25. The method of claim 21, wherein each fluoroscopic frame contains a radiopaque calibration device comprising a plurality of radiopaque markers at known three-dimensional coordinates, and the method further comprises computing per-frame intrinsic and extrinsic camera parameters from detected marker features by establishing two-dimensional-to-three-dimensional point correspondences and solving the perspective-n-point problem.

26. The method of claim 25, wherein radiopaque calibration device artifacts are digitally removed from each frame prior to producing the two-dimensional representations.

27. The method of claim 21, further comprising training the trained three-dimensional deep learning network on pairs of CT volumes and simulated digitally reconstructed radiographs generated via polychromatic X-ray simulation with clinically matched tube voltage, tube current, and exposure time distributions.

28. The method of claim 21, wherein the fluoroscopic image acquisition system comprises a fixed bi-plane fluoroscopy system with two orthogonal X-ray sources and detectors acquiring synchronized images simultaneously.

29. A computer program product comprising a non-transitory computer-readable medium storing instructions that, when executed by a processor coupled to a graphics processing unit, cause the processor to perform a method comprising:
(a) receiving, from a fluoroscopic image acquisition system comprising a radiation source and a radiographic image detector, a sequence of N fluoroscopic frames, wherein N>4, captured during one or more continuous translational sweeps along an extended anatomical region of a patient, the fluoroscopic frames in any image format including native digital or digitized analog video, together with six-degree-of-freedom positional data generated by a spatial tracking system tracking a first fiducial marker mounted to the fluoroscopic image acquisition system and a second fiducial marker affixed to the patient proximate the extended anatomical region for at least one reference frame;
(b) producing, from the N fluoroscopic frames, one or more two-dimensional representations, each being a raw fluoroscopic image, a composited panoramic image, or learned feature maps from a trained two-dimensional deep learning network, and back-projecting the two-dimensional representations into one or more three-dimensional voxel grids using projection matrices derived from the positional data;
(c) producing a volumetric output by processing the back-projected representations through a trained three-dimensional deep learning network, the volumetric output spanning a spatial extent, in at least one dimension, exceeding the field of view of any single fluoroscopic frame; and
(d) registering the volumetric output to a patient coordinate system using spatial tracking measurements of the first and second fiducial markers, the registration being independent of any preoperative volumetric imaging study of the patient and any manual intraoperative landmark-correspondence step.

30. The computer program product of claim 29, wherein the instructions further cause the processor to, prior to back-projection, composite sequential fluoroscopic frames from each sweep orientation into a panoramic image by determining inter-frame spatial relationships from image content by detecting and matching image features in overlapping regions between adjacent frames, composing a cumulative chain of inter-frame geometric transformations from a single tracked reference pose, and back-projecting the panoramic image into a single extended three-dimensional voxel grid for reconstruction in one pass.
